(19) United States
(12) Patent Application Publication
(10) Pub. No.: US 2015/0152457 A1
(43) Pub. Date: Jun. 4, 2015

(54) DIRECT STARCH TO FERMENTABLE SUGAR

(71) Applicant: Danisco US Inc., Palo Alto, CA (US)

(72) Inventors: Collette Michelle Blake, North Liberty, IA (US); Gang Duan, Shanghai (CN); Douglas Ko, Milpitas, CA (US); Floor K. Kooy, Leiden (NL); Sung Ho Lee, North Liberty, IA (US); Michael Jay Pepsin, Castro Valley, CA (US); Ying Qian, Wuxi (CN); Matthew T. Reboli, Sunnyvale, CA (US); Vivek Sharma, Fremont, CA (US); Jayarama K. Shetty, Pleasanton, CA (US); Bruce A. Strohm, Beloit, WI (US); Paula Johanna Maria Teunissen, Saratoga, CA (US); Hongxian Xu, Wuxi City (CN)

(73) Assignee: Danisco US Inc., Palo Alto, CA (US)

(21) Appl. No.: 14/479,683

(22) PCT Filed: Mar. 13, 2013
(86) PCT No.: PCT/US2013/030980

§ 371 Date: Sep. 8, 2014

Related U.S. Application Data
(60) Provisional application No. 61/618,533, filed on Mar. 30, 2012.

(51) Int. Cl. C12P 19/14; C12P 19/02

(52) U.S. Cl. CPC C12P 19/14; C12P 19/02

(57) ABSTRACT

Provided herein are compositions and methods related to the direct conversion of the starch in a ground or fractionated grain into a fermentable sugar feedstock capable of serving as a carbon source for the industrial production of one or more products by a fermenting organism. Such conversions may be performed at temperatures at or below the initial gelatinization temperature of the starch present in the grain and may utilize one or more isolatable endogenous enzymes present in certain unrefined grains.

### CROSS-REFERENCE TO RELATED APPLICATIONS

This application claims benefit to U.S. Provisional patent application 61/618,533, filed on Mar. 30, 2012, the contents of which are hereby incorporated by reference in its entirety.

### FIELD OF THE INVENTION

This disclosure is directed towards compositions and methods related to the direct conversion of starch in a ground or fractionated grain into a fermentable sugar feedstock capable of serving as a carbon source for the industrial production of one or more products by a fermenting organism.

### BACKGROUND OF THE INVENTION

A number of agricultural crops are viable candidates for the conversion of starch to fermentable feed stock. Such fermentable feedstocks can be fed to various microbes to produce a variety of biochemicals. Typically, corn is used as the primary starch source for producing fermentable glucose. However other high-starch content sources like sorghum, wheat, barley, rye and cassava are beginning to gain more attention as a viable feedstock for the industrial production of biochemicals and fuel. The conventional process for producing a fermentable high glucose syrup feedstock from insoluble starch involves heating whole ground grain or starch slurry to temperatures in excess of 95° C. in the presence of alpha amylase (a process known as “liquefaction”), followed by cooling, pH adjustment, and subsequent glucoamylase hydrolysis (otherwise known as “saccharification”). Such processes can produce fermentable feed stocks containing greater than 90% glucose. However, these conventional approaches are highly energy-intensive.

Various industrial processes have been adopted by the starch sweetener industry for enzyme-mediated liquefaction (see, e.g. U.S. Pat. No. 5,322,778). Some of these processes are performed at lower temperatures with relatively low steam requirements (e.g., 105-110° C. for 5-8 min) while others are high temperature processes (e.g., 148° C.+/−5° C. for 8-10 sec), resulting in improved gelatinization of starch granules leading to improved filtration characteristics and quality of the liquefied starch substrate (Shetty, et al., (1988) Cereal Foods World 33:929-934). Further advances in the liquefaction process have been demonstrated by multiple additions of thermostable alpha amylases in the pre/post jet cooking step, which results in significant improvements with respect to yield loss, processing costs, energy consumption, pH adjustments, temperature thresholds, calcium requirements and levels of retrograded starch.

The drastic conditions required for liquefaction (e.g. high temperature and pH), negatively affect the bioconversion efficiency of whole ground grains into feedstocks, resulting in the loss of fermentable sugars, production of Maillard reaction products, destruction of essential nutrients (e.g., free sugars, free amino acids, minerals, vitamins), deactivation of native beneficial enzymes (e.g. amylases, proteases, and phytases) and/or cross-linking or condensation of cellular components such as tannins and starch proteins (Wu et al., Cereal Chem., 2007, 84:130-13). Furthermore, significant energy costs are associated with high-temperature cooking of grain to aid in enzymatic digestion. In addition, in the context of the dry grind process for ethanol, the incomplete gelatinization during high temperature cooking (i.e. temperatures exceeding the starch gelatinization temperature) for solubilizing granular starch in ground whole grain is believed to be the principle reason for lower digestibility by alpha amylases. The digestibility of starch is also negatively affected by starch-lipid and starch-protein complexes formed during the interaction of reactive proteins and lipids with starch at liquefaction conditions (Zhang & Hamaker, Cereal Chem., 1998, 75:710-713). Another major problem associated with liquefaction at high temperature is high viscosity due to the rapid swelling of starch and non-starch polysaccharide components such as beta-glucan.

Due to increasing concern for the environment and the need to limit greenhouse gases, sources of renewable energy are gaining wide-spread attention. The recent development of no-cook processes using enzymes capable of hydrolyzing granular starch directly into fermentable glucose have made significant improvements in the energy required for ethanol production (see, e.g., U.S. Pat. No. 7,037,704; U.S. Patent Application Publication Nos.: 2003/0180900 A1, 2006/0121589 A1, and US 2004/0234649 A1; and International Patent Application Publication No.: 2004/081193 A2). However, these processes require extensive milling for fine grind particles, longer fermentation times, and potential risk of microbial infection.

Another major problem associated with current processes for the production of high fermentable glucose syrup (such as syrups with greater than 96% fermentable sugar) is loss of glucose yield due to the production of the reversion reaction product, isomaltose (6 O-α-D-glucopyranosyl-α [1-6]-α-D-Glucopyranoside). This reversion reaction can be catalyzed by glucoamylases during saccharification. Many microorganisms used for the production of valuable biochemicals cannot ferment isomaltose, and such approaches also result in downstream processing losses during recovery, and yield losses. What is needed, therefore, is a low temperature process for the direct production of fermentable feedstocks from ground or fractionated grain that avoids the drawbacks associated with currently available no-cook enzymatic starch hydrolysis processes and which contains reduced amounts of reversion products, such as isomaltose.

The invention described herein addresses these problems and provides additional benefits as well.

All patents, patent applications, publications, documents, nucleotide and protein sequence database accession numbers, the sequences to which they refer, and articles cited herein are all incorporated herein by reference in their entireties.

### BRIEF SUMMARY OF THE INVENTION

The invention provided herein discloses, inter alia, compositions and methods for a low temperature process for hydrolyzing granular starch within ground whole or fractionated grains into highly fermentable sugars.

Accordingly, in some aspects, provided herein are methods for making a fermentable sugar feedstock, the method comprising treating an aqueous slurry of ground or fractionated grain with an alpha amylase and a glucoamylase to produce the fermentable sugar feedstock; wherein the treatment is at a temperature at or below the initial gelatinization temperature of the starch in the grain; wherein the concentration of the alpha amylase is between about 5 to about 20 AAU/gds; and wherein the fermentable sugar feedstock comprises a higher concentration of DP-2 saccharides in comparison to fermentable sugar feedstocks that are not made by treating an aqueous slurry of ground or fractionated grain with a starch solubilizing alpha amylase and a glucoamylase, wherein the treatment is at a temperature below the initial gelatinization temperature of the grain, and wherein the concentration of the alpha amylase is between about 5 to about 20 AAU/gds. In some aspects, the treatment is at a temperature of about 0 to about

30° C. below the initial gelatinization temperature of the starch in the grain. In some aspects, the concentration of alpha amylase is about 6 AAU/g ds to about 10 AAU/g ds. In some aspects, the ground or fractionated grain is selected from the group consisting of: corn, corn endosperm, milo, rice, and any combination thereof. In some aspects, greater than about 90% of the starch from the ground or fractionated grain is solubilized. In some aspects, the solubilized starch comprises greater than about 90% fermentable sugars. In some aspects, the alpha amylase is derived from a Bacillus spp. In some aspects, the alpha amylase is selected from the group consisting of SPEZYME® XTRA, SPEZYME® Alpha, SPEZYME® RSL, Liquozyme SC, and Fuelzyme. In some aspects of any of the aspects above, the aqueous slurry with one or more enzymes selected from the group consisting of: cellulases, hemicellulases, pullulanases, pectinases, phytases, and proteases. In some aspects of any of the aspects above, the method further comprises treating the aqueous slurry with an acid fungal alpha amylase. In some aspects of any of the aspects above, the treatment is at a temperature of about 55 to about 65° C. In some aspects of any of the aspects above, the concentration of glucoamylase is about 0.025 GAU/g ds to about 0.075 GAU/g ds. In some aspects of any of the aspects above, the concentration of glucoamylase is about 0.075 GAU/g ds to about 0.2 GAU/g ds. In some aspects of any of the aspects above, the DP-2 saccharides comprise kojibiose and/or nigerose. In some aspects of any of the aspects above, the method further comprises using the fermentable sugar feedstock as a carbon source for the industrial production of one or more products by a fermenting microorganism. In some aspects, the fermenting microorganism is a yeast or a bacteria. In some aspects of any of the aspects above, the product is an enzyme. In some aspects of any of the aspects above, the enzyme is used in the processing of grain, as an additive or in the preparation of food, as an additive or in the preparation of animal feed, as an ingredient in a detergent or cleaning agent, in the processing of textiles, or in the processing of pulp for the manufacture of paper. In some aspects, the product is a fermentation product selected from the group consisting of ethanol, lactic acid, gluconic acid, butanol, succinic acid, citric acid, monosodium glutamate, sodium gluconate, calcium gluconate, potassium gluconate, glucono delta lactone, sodium erythorbate, itaconic acid, ketones, amino acids, glutamic acid (sodium monoglutamate), penicillin, tetracyclin, enzymes, vitamins, and hormones.

In some aspects, provided herein are methods for making a fermentable sugar feedstock, the method comprising treating a refined granular starch with a starch solubilizing alpha amylase, a glucoamylase, and an enzyme-containing extract to produce the feedstock, wherein, the treatment is at a temperature at or below the initial gelatinization temperature of the starch in the grain; wherein the concentration of the alpha amylase is between about 5 to about 20 AAU/gds; wherein the enzyme-containing extract is from a grain; and wherein the fermentable sugar feedstock comprises a higher concentration of DP-2 saccharides in comparison to fermentable sugar feedstocks that are not made by treating a refined granular starch with a starch solubilizing alpha amylase, a glucoamylase, and an enzyme-containing extract wherein the treatment is at a temperature at or below the initial gelatinization temperature of the grain, wherein the concentration of the alpha amylase is between about 5 to about 20 AAU/gds, and wherein the enzyme-containing extract is derived from a grain. In some aspects, the enzyme-containing extract is derived from a grain selected from the group consisting of: whole ground corn, corn endosperm, whole ground milo, whole ground rice, and any combination thereof. In some aspects, the refined granular starch is refined corn starch. In some aspects, the treatment is at a temperature of about 0 to about 30° C. below the initial gelatinization temperature of the starch in the grain. In some aspects, the concentration of alpha amylase is about 6 AAU/g ds to about 10 AAU/g ds. In some aspects, the ground or fractionated grain is selected from the group consisting of: corn, corn endosperm, milo, rice, and any combination thereof. In some aspects, greater than about 90% of the starch from the ground or fractionated grain is solubilized. In some aspects, the solubilized starch comprises greater than about 90% fermentable sugars. In some aspects, the alpha amylase is derived from a Bacillus spp. In some aspects, the alpha amylase is selected from the group consisting of SPEZYME® XTRA, SPEZYME® Alpha, SPEZYME® RSL, Liquozyme SC, and Fuelzyme. In some aspects of any aspect described above, the method further comprises treating the aqueous slurry with one or more enzymes selected from the group consisting of: cellulases, hemicellulases, pullulanases, pectinases, phytases, and proteases. In some aspects of any aspect described above, the method further comprises treating the aqueous slurry with an acid fungal alpha amylase. In some aspects of any aspect described above, the treatment is at a temperature of about 55 to about 65° C. In some aspects of any aspect described above, the concentration of glucoamylase is about 0.025 GAU/g ds to about 0.075 GAU/g ds. In some aspects of any aspect described above, the concentration of glucoamylase is about 0.075 GAU/g ds to about 0.2 GAU/g ds. In some aspects of any aspect described above, the DP-2 saccharides comprise kojibiose and/or nigerose. In some aspects of any aspect described above, wherein the method further comprises using the fermentable sugar feedstock as a carbon source for the industrial production of one or more products by a fermenting microorganism. In some aspects, the fermenting microorganism is a yeast or a bacteria. In some aspects, the product is an enzyme. In some aspects, the enzyme is used in the processing of grain, as an additive or in the preparation of food, as an additive or in the preparation of animal feed, as an ingredient in a detergent or cleaning agent, in the processing of textiles, or in the processing of pulp for the manufacture of paper. In some aspects, the product is a fermentation product selected from the group consisting of ethanol, lactic acid, gluconic acid, butanol, succinic acid, citric acid, monosodium glutamate, sodium gluconate, calcium gluconate, potassium gluconate, glucono delta lactone, sodium erythorbate, itaconic acid, ketones, amino acids, glutamic acid (sodium monoglutamate), penicillin, tetracyclin, enzymes, vitamins, and hormones.

Provided herein are methods for making a fermentable sugar feedstock having a reduced concentration or amount of DP-2 saccharides, the method comprising: (a) inactivating endogenous enzyme activity in a whole or fractionated grain; and (b) treating the whole or fractionated grain with a starch solubilizing alpha amylase and a glucoamylase to produce the fermentable sugar feedstock, wherein the treatment is at a temperature at or below the initial gelatinization temperature of the starch in the grain; wherein the concentration of the alpha amylase is between about 5 to about 20 AAU/gds; and wherein the fermentable sugar feedstock comprises a decreased concentration of DP-2 saccharides in comparison to fermentable sugar feedstocks that are not made by inactivating endogenous enzyme activity in a whole or fractionated grain and treating the whole or fractionated grain with a starch solubilizing alpha amylase and a glucoamylase, wherein the treatment is at a temperature at or below the initial gelatinization temperature of the starch in the grain, and wherein the concentration of the alpha amylase is between about 5 to about 20 AAU/gds. In some aspects, the whole or fractionated grain is whole crown corn or corn endosperm. In some aspects, endogenous enzyme activity is inactivated by exposing the whole or fractionated grain to a pH of about 1 to about 3. In some aspects, the reduced concentration of DP-2 saccharides comprises reduced concentration of kojibiose and/or nigerose. In some aspects, the treatment is at a temperature of about 0 to about 30° C. below the initial gelatinization temperature of the starch in the grain. In some aspects, the concentration of alpha amylase is about 6 AAU/g ds to about 10 AAU/g ds. In some aspects, the ground or fractionated grain is selected from the group consisting of: corn, corn endosperm, milo, rice, and any combination thereof. In some aspects, greater than about 90% of the starch from the ground or fractionated grain is solubilized. In some aspects, the solubilized starch comprises greater than about 90% fermentable sugars. In some aspects, the alpha amylase is derived from a Bacillus spp. In some aspects, the alpha amylase is selected from the group consisting of SPEZYME® XTRA, SPEZYME® Alpha, SPEZYME® RSL, Liquozyme SC, and Fuelzyme. In some aspects of any of the aspects above, the method further comprises treating the aqueous slurry with one or more enzymes selected from the group consisting of: cellulases, hemicellulases, pullulanases, pectinases, phytases, and proteases. In some aspects of any of the aspects above and herein, the method further comprises treating the aqueous slurry with a phytase. In some aspects of any of the aspects above, the method further comprises treating the aqueous slurry with an acid fungal alpha amylase. In some aspects of any of the aspects above, the treatment is at a temperature of about 55 to about 65° C. In some aspects of any of the aspects above, the concentration of glucoamylase is about 0.025 GAU/g ds to about 0.075 GAU/g ds. In some aspects of any of the aspects above, the concentration of glucoamylase is about 0.075 GAU/g ds to about 0.2 GAU/g ds. In some aspects of any of the aspects above, the DP-2 saccharides comprise kojibiose and/or nigerose. In some aspects of any of the aspects above, wherein the method further comprises using the fermentable sugar feedstock as a carbon source for the industrial production of one or more products by a fermenting microorganism. In some aspects, the fermenting microorganism is a yeast or a bacteria. In some aspects, the product is an enzyme. In some aspects, the enzyme is used in the processing of grain, as an additive or in the preparation of food, as an additive or in the preparation of animal feed, as an ingredient in a detergent or cleaning agent, in the processing of textiles, or in the processing of pulp for the manufacture of paper. In some aspects, the product is a fermentation product selected from the group consisting of ethanol, lactic acid, gluconic acid, butanol, succinic acid, citric acid, monosodium glutamate, sodium gluconate, calcium gluconate, potassium gluconate, glucono delta lactone, sodium erythorbate, itaconic acid, ketones, amino acids, glutamic acid (sodium monoglutamate), penicillin, tetracyclin, enzymes, vitamins, and hormones.

In some aspects, provided herein are methods for making a fermentation product, the method comprising: (a) providing a fermenting organism with the fermentable feedstock produced according to the methods of claim 1, 2, or 3; and, (b) making a product. In some aspects, the product is a fermentation product selected from the group consisting of ethanol, lactic acid, gluconic acid, butanol, and succinic acid. In some aspects, the product is selected from the group consisting of glycerol, 1,3-propanediol, gluconate, 2-keto-D-gluconate, 2,5-diketo-D-gluconate, 2-keto-L-gulonic acid, amino acids, gluconic acid, citric acid, monosodium glutamate, sodium gluconate, calcium gluconate, potassium gluconate, glucono delta lactone, sodium erythorbate, itaconic acid, ketones, glutamic acid, penicillin, tetracyclin, vitamins, and hormones and derivatives thereof. In some aspects, the product is an enzyme. In some aspects, the enzyme is selected from the group consisting of glucoamylases, amylases, pullulanases, cellulases, xylanases, hemicellulases, proteases, phytases, lipases, esterases, cutinases, pectinases, oxidases, catalases, transferases, glucose isomerases, glucosidases, isomerases, and one or more enzymes involved in the anabolism of one or more amino acids. In some aspects, the fermenting microorganism is a yeast a bacteria, or a fungus. In some aspects, the fermenting microorganism is a yeast selected from the group consisting of a Saccharomyces spp., a Pichia spp., a Candida spp., a Hansenula spp., a Kluyveromyces spp., a Kluyveromyces spp., and a Schizosaccharomyces spp. In some aspects, the fermenting microorganism is a bacteria selected from the group consisting of an Arthrobacter spp., an Escherichia spp., a Zymomonas spp., a Brevibacterium spp., a Clostridium spp., an Aerococcus spp., a Bacillus spp., a Carbobacterium spp., a Corynebacterium spp., an Enterococcus spp., an Erysipelothrix spp., a Gemella spp., a Geobacillus spp., a Globicatella spp., a Lactobacillus spp., a Lactococcus spp., a Leuconostoc spp., a Pediococcus spp., a Streptococcus spp., a Tetragenococcus spp., an Actinobacillus spp., and a Vagococcus spp. In some aspects, the fermenting microorganism is a fungus. In some aspects, the fungus is a Rhizopus spp.

### BRIEF DESCRIPTION OF THE DRAWINGS

FIG. 1 depicts the process for obtaining a fermentable sugar feedstock from starch at temperatures at or below the initial gelatinization temperature of starch in a grain.

FIG. 2 depicts DP sugar profile of the sugar syrup used in enzyme production experiments.

FIG. 3 depicts enzyme activity vs. fermentation time for a representative experiment illustrating the production of a protease derived from Bacillus amyloliquefaciens expressed in Bacillus subtilis which utilizes a DSTFS feedstock produced according to the methods described herein as a carbon source.

FIG. 4 depicts enzyme activity vs. fermentation time for a representative experiment illustrating the production of a phytase derived from Buttiauxella expressed in Trichoderma reesei which utilizes a DSTFS feedstock produced according to the methods described herein as a carbon source.

FIG. 5 depicts the percent of starch solubilization versus incubation time for a representative experiment illustrating the effect of phytase added to corn in a granular starch hydrolysis process.

FIG. 6 depicts a zoomed-in view of FIG. 5.

FIG. 7 depicts the percent of glucose (DP1) solubilization versus incubation time for a representative experiment illustrating the effect of phytase with alpha amylase and gluco-amylase in a granular starch hydrolysis process.

FIG. 8 depicts a zoomed-in view of FIG. 7.

FIG. 9 depicts the percent of starch solubilization versus incubation time for a representative experiment illustrating the effect of phytase with gluco-amylase in a granular starch hydrolysis process.

FIG. 10 depicts a zoomed-in view of FIG. 9.

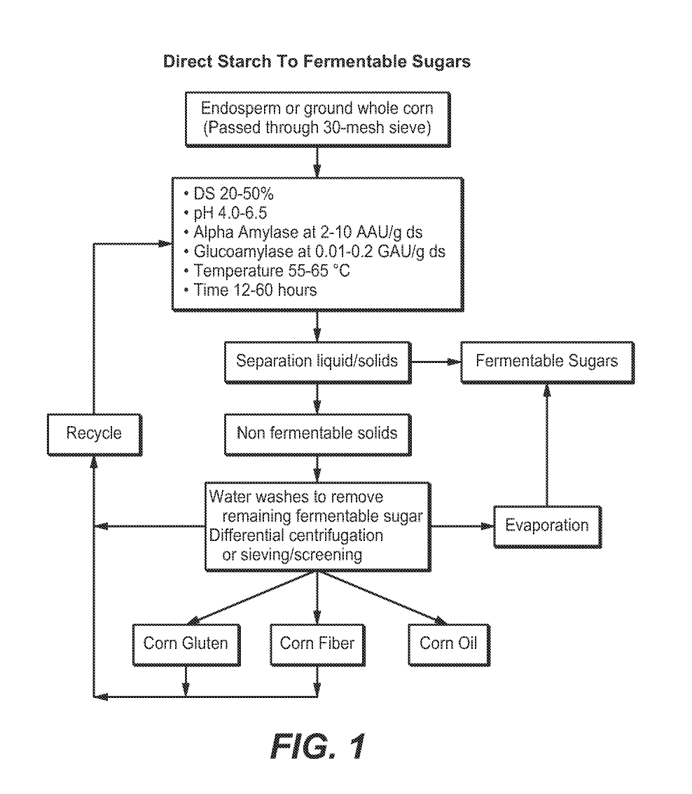

### DETAILED DESCRIPTION

The invention provides, inter alia, compositions and methods for the low-temperature production of sugar feedstocks that are useful as fermentable carbon sources for the industrial production of one or more products by a fermenting microorganism.

Dry grind and wet mill grain processes traditionally cook grains and other starch feedstocks with thermostable enzymes to begin the process of converting insoluble starch to fermentable sugars. In an exemplary dry grind process, the entire corn kernel or other starchy grain can be first ground and then processed without separating the various cellular components of the grain. In general, two enzymatic steps can be involved in the hydrolysis of starch to glucose: liquefaction followed by saccharification. During liquefaction, insoluble starch granules can be slurried in water, gelatinized with heat, and hydrolyzed by a thermostable alpha amylase (EC.3.2.1.1, alpha (1-4)-glucan glucanohydrolase), for example, a Bacillus species, in the presence of added calcium. Bacterially derived thermostable alpha amylases are used to first liquefy the starch at high temperature (these can be greater than 95° C.) at an acidic pH (e.g., pH 5.4-6.5) to a low DE (dextrose equivalent) soluble starch hydrolysate. Saccharification further hydrolyzes the soluble low DE dextrins to glucose via an enzyme having glucoamylase (EC 3.2.1.3, alpha (1,4)-glucan glucohydrolase) activity. Commercial glucoamylases are primarily derived from fungal sources, for example Aspergillus, Trichoderma, Rhizopus, Talaromyces and Tramates species. The high glucose syrup may then be used as feedstock to be converted into other commercially important end-products, such as enzymes, proteins, fructose, sorbitol, ethanol, butanol, lactic acid, ascorbic acid intermediates, succinic acid, and 1,3 propane diol.

The present invention provides for a low-temperature process for the efficient production of fermentable feedstocks that avoids the extensive milling, long fermentation times, and risk of microbial infection associated with currently available low temperature processes. The inventors have discovered, inter alia, that the starch present in unrefined grains can be hydrolyzed to yield feedstocks containing up to about 98% (such as up to about 90%, 91%, 92%, 93%, 94%, 95%, 96%, or 97%) fermentable sugars at temperatures at or below the initial gelatinization temperature of the starch in the grain. Without being bound to theory, and in one aspect, one or more endogenous enzyme(s) present in certain grains can effectively hydrolyze a starch source into a fermentable feedstock with a distinct DP2 saccharide profile when used in combination with high doses of exogenous alpha amylase and glucoamylase. The methods of the present application, therefore, represent an improvement over what has previously been practiced in the art, in that starch hydrolysis can be performed relatively rapidly on course ground or fractionated grains and at temperatures significantly below those required for most dry mill or wet mill processes that require high temperatures for starch hydrolysis. Consequently, the methods of the present invention require significantly less energy for the hydrolysis of starch into fermentable feedstocks and do not require extensive grain processing and/or long fermentations which can increase the risk of microbial feedstock contamination.

### General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994). Singleton et al., “Dictionary of Microbiology and Molecular Biology” 2nd ed., J. Wiley & Sons (New York, N.Y. 1994), and Baltz et al., “Manual of Industrial Microbiology and Biotechnology” 3^{rd} ed., (Washington, D.C.: ASM Press, 2010), provide one skilled in the art with a general guide to many of the terms used in the present application.

### DEFINITIONS

As used herein the term “starch” refers to any material comprised of the complex polysaccharide carbohydrates of plants, comprised of amylose and/or amylopectin with the formula (C₆H₁₀O₅)ₓ, wherein X can be any number. In particular, the term refers to any plant-based material including but not limited to grains, grasses, tubers and roots and more specifically wheat, barley, corn, rye, rice, sorghum (milo), molasses, legumes, cassava, millet, potato, sweet potato, sugar cane, and tapioca.

The term “granular starch” refers to uncooked (raw) starch, which has not been subjected to gelatinization.

The term “starch gelatinization” means solubilization of a starch molecule to form a viscous suspension.

“Initial gelatinization temperature” refers to the lowest temperature at which gelatinization of a starch substrate begins. The exact temperature can be readily determined by the skilled artisan and depends upon the specific starch substrate. Initial gelatinization temperature may further depend on the particular variety of plant species from which the starch is obtained and the growth conditions. According to the present teachings, the initial gelatinization temperature of a given starch is the temperature at which birefringence is lost in 5% of the starch granules using the method described by Gorinstein & Lii, Starch/Stark, 1992, 44(12):461-466. The initial starch gelatinization temperature ranges for a number of granular starches which may be used in accordance with the processes herein include barley (52-59° C.), wheat (58-64° C.), rye (57-70° C.), corn (62-72° C.), high amylose corn (67-80° C.), rice (68-77° C.), sorghum (68-77° C.), potato (58-68° C.), tapioca (59-69° C.) and sweet potato (58-72° C.) (Swinkels, pg. 32-38 in Starch Conversion Technology, Van Beynum et al., eds. (1985; Marcel Dekker Inc., New York) and The Alcohol Textbook 3^{rd} Ed. A Reference for the Beverage, Fuel and Industrial Alcohol Industries, Jacques et al., eds. (1999; Nottingham University Press, UK)). Gelatinization involves melting of crystalline areas, hydration of molecules and irreversible swelling of granules. The gelatinization temperature occurs in a range for a given grain because crystalline regions vary in size and/or degree of molecular order or crystalline perfection. Starch Hydrolysis Products: Worldwide Technology, Production, and Applications (Shenck and Hebeda, eds. (1992; VCH Publishers, Inc., New York) at p. 26.

The term “DE” or “dextrose equivalent” is an industry standard for measuring the concentration of total reducing sugars, calculated as D-glucose on a dry weight basis. Unhydrolyzed granular starch has a DE that is essentially 0 and D-glucose has a DE of 100.

The term “endosperm” refers to the grain after separating the germ and crude fiber fractions.

The term “fermentation broth” refers to fermentation medium containing the end products after fermentation.

The term “direct starch to fermentable sugars” (“DSTFS”) refers to a process whereby granular starch hydrolysates are formed using direct conversion of granular starch to fermentable sugars according to of the processes disclosed in the present teachings.

The term “DP” refers to degree of polymerization to the number (n) of anhydroglucopyranose units in a given saccharide. Non-limiting examples of DP1 saccharides are the monosaccharides, such as glucose and fructose. Non-limiting examples of DP2 saccharides are disaccharides such as maltose, isomaltose and sucrose. DP4⁺(>DP3) denotes polymers with a degree of polymerization of greater than 3.

The term “fermentable sugar” refers to the sugar composition containing DP1 and DP2.

The term “ds or DS” refers to dissolved solids and/or dry substance in a solution.

The term “starch-liquefying enzyme” refers to an enzyme that affects the hydrolysis or breakdown of granular starch. Exemplary starch liquefying enzymes include alpha amylases (E.C. 3.2.1.1).

The term “amylases” refer to enzymes that catalyze the hydrolysis of starches.

The term “alpha-amylase (E.C. class 3.2.1.1)” refers to enzymes that catalyze the hydrolysis of alpha-1,4-glucosidic linkages. These enzymes have also been described as those effecting the exo or endohydrolysis of 1, 4-α-D-glucosidic linkages in polysaccharides containing 1, 4-α-linked D-glucose units. Another term used to describe these enzymes is glycogenase. Exemplary enzymes include alpha-1,4-glucan 4-glucanohydrase glucanohydrolase.

The term “glucoamylase” refers to the amyloglucosidase class of enzymes (EC.3.2.1.3, glucoamylase, alpha-1, 4-D-glucan glucohydrolase). These are exo-acting enzymes, which release glucosyl residues from the non-reducing ends of amylose and amylopectin molecules. The enzymes also hydrolyze alpha-1,6 and alpha-1,3 linkages although at much slower rates than alpha 1,4 linkages. Glucoamylases (E.C. 3.2.1.3) are enzymes that remove successive glucose units from the non-reducing ends of starch. The enzyme can hydrolyze both linear and branched glucosidic linkages of starch, amylose and amylopectin.

The term “hydrolysis of starch” refers to the cleavage of glucosidic bonds with the addition of water molecules.

The term “contacting” refers to the placing of the respective enzymes in sufficiently close proximity to the respective substrate to enable the enzymes to convert the substrate to the end product. Those skilled in the art will recognize that mixing solutions of the enzyme with the respective substrates can effect contacting.

The term “fermentable sugar index” refers to the number calculated by using the following formula: ((% DP1+% DP2)/% DP3+% Hr. Sugars)*100.

As used herein, the terms “minimal medium” or “minimal media” refer to growth medium containing the minimum nutrients possible for cell growth, generally without the presence of amino acids. Minimal medium can contain: (1) a carbon source for microbial growth; (2) various salts, which can vary among microbial species and growing conditions; and (3) water. The carbon source can vary significantly, from simple sugars like glucose to more complex hydrolysates of other biomass, such as yeast extract. The salts generally provide essential elements such as magnesium, nitrogen, phosphorus, and sulfur to allow the cells to synthesize proteins and nucleic acids. Minimal medium can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. Medium can be supplemented with other compounds as necessary to select for desired physiological or biochemical characteristics, such as particular amino acids and the like.

The term “fermenting organism” or “fermenting microorganism” refers to any organism, such as, but not limited to bacterial and fungal organisms, (e.g. yeast and filamentous fungi), suitable for producing a desired fermentation product. Fermenting organisms possess the ability to ferment, (such as to change or convert) sugars, such as glucose, xylose, maltose, fructose, xylose, arabinose and/or mannose, directly or indirectly into a desired fermentation product. Examples of fermenting organisms include fungal organisms, such as yeast, and bacterial organisms such as, but not limited to, E. coli, Bacillus spp., Zymomonas spp., and Clostridium spp. In some aspects, the fermenting organism can be a mircrobe.

Unless defined otherwise herein, all technical and scientific terms used herein have the same meaning as commonly understood by one of ordinary skill in the art to which this invention pertains.

As used herein, the singular terms “a,” “an,” and “the” include the plural reference unless the context clearly indicates otherwise.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Starch-Containing Material

Starch-containing materials useful for practicing the methods of the present invention include any starch-containing material. Preferred or exemplary starch-containing material may be obtained from wheat, corn, rye, sorghum (milo), rice, millet, barley, triticale, cassava (tapioca), potato, sweet potato, sugar beets, sugarcane, and legumes such as soybean and peas or any combination thereof. Plant material may include hybrid varieties and genetically modified varieties (e.g. transgenic corn, barley or soybeans comprising one or more heterologous genes). Any part of the plant may be used as a starch-containing material, including but not limited to, plant parts such as leaves, stems, hulls, husks, tubers, cobs, grains and the like.

In some aspects, only the grain product of one or more cereals may be used as a starch-containing material. Cereal grains have three components: the endosperm, germ, and bran. In their natural form (for example, “whole” grain), they are a rich source of vitamins, minerals, carbohydrates, fats, oils, and protein. Grains which are ground whole (i.e., whole ground grains) contain ground endosperm, grain and bran. However, grains may also be fractionated by the separation of the bran and germ from the endosperm, which is mostly carbohydrate and lacks the majority of the other nutrients. Non-limiting examples of whole grains which can be used in the methods disclosed herein include corn, wheat, rye, barley, milo and combinations thereof.

In other aspects, starch-containing material may be obtained from coarsely ground or fractionated cereal grains including fiber, endosperm and/or germ components. Methods for fractionating plant material, such as corn and wheat, are known in the art (Alexander, 1987, “Corn Dry Milling: Process, Products, and Applications,” in Corn Chemistry and Technology, (Watson & Ramstead eds., American Association of Cereal Chemists, Inc., pgs. 351-376; U.S. Pat. No. 6,899,910, the disclosures of which are incorporated herein by reference). Coarsely ground or fractionated starch-containing material obtained from different sources may be mixed together to obtain material used in the processes of the invention (e.g. corn and milo or corn and barley).

In another aspect, a refined grain may be used in the methods described herein when used in combination with an endogenous enzyme fraction derived from specific grains, such as the endogenous enzyme fractions described below. Refined grains, in contrast to whole grains, refer to grain products such as grain flours that have been modified from their natural composition and are essentially pure starch. Such modification can include, but is not limited to, the mechanical removal of bran and germ, either through grinding or selective sifting. Examples of refined grain include, but are not limited to, corn starch, wheat starch, and rice starch.

Milling Starch-Containing Material

In some aspects, starch-containing material may be prepared by means such as milling. Two general milling processes include wet milling or dry milling. In dry milling for example, the whole grain is milled and used in the process. In wet milling the grain is separated (e.g. the germ from the meal). In particular, means of milling whole cereal grains are well known and include the use of hammer mills and roller mills. Methods of milling are well known in the art and reference is made to The Alcohol Textbook: A Reference for the Beverage, Fuel and Industrial Alcohol Industries, 3rd edition, (Jacques et al., Eds, 1999; Nottingham University Press, chapters 2 and 4, the contents of which are hereby incorporated by reference), the contents of which are incorporated herein by reference. In some aspects, the milled grain used in the process has a particle size such that more than about 50% (for example, more than about 55%, 60%, 65%, 70%, 75%, 80%, 85%, 90% or 95%) of the material will pass through a sieve with a 500 micron opening (see, for example, International Patent Application Publication No.: WO2004/081193, the contents of which are incorporated by reference).

Slurries of Starch-Containing Material

In some aspects of the methods provided herein, an aqueous slurry of coarsely ground or fractionated grain (such as any of the milled grains described herein) may be formed prior to liquefaction and saccharification of the starch contained within. The milled starch-containing material is normally screened to a specified sieve size (such as, but not limited to, a 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, or 500 μm sieve) and is combined with water resulting in aqueous slurry.

In some aspects, the dissolved solids (DS) present in the slurry is between about 20-50%, inclusive, including any percentage in between these values. In other aspects, the DS of the slurry between about 25-50%, about 30-50%, about 35-50%, about 40-50%, or about 45-50%, inclusive. In yet other aspects, the DS of the slurry is between about 20-45%, about 20-40%, about 20-35%, about 20-30%, or about 20-25%, inclusive. In a further aspect, the DS of the slurry is between about 25-45% or about 30-40%, inclusive. The pH of the slurry may range from neutral to acidic. In some aspects, the pH of the slurry is between about 4.0-6.5, inclusive, including any number in between these values. In other aspects, the pH of the slurry is between about 4.2-6.2, about 4.5-6.0, about 4.5-5.5, about 5.5-6.0, or about 5.0-6.0, inclusive. In other aspects, the slurry can comprise between about 15 to 55% ds w/w (e.g., between about 20 to 50%, 25 to 50%, 25 to 45%, 25 to 40%, or 20 to 35% ds), inclusive.

### Enzymes

The methods described herein can employ one or more enzymes to assist in the liquefaction and saccharification of starch at temperatures below that of the gelatinization temperature of starch in grain.

Alpha Amylases

In some aspects, the methods provided herein use at least one alpha amylase for the production of a fermentable feedstock. Specifically, alpha amylases are employed in the methods disclosed herein for the liquefaction of raw or granular starch into viscous short chain dextrins. Alpha amylase is an enzyme having an E.C. number of E.C. 3.2.1.1-3 (including, for example, E.C. 3.2.1.1) that hydrolyses the alpha-bonds of large alpha-linked polysaccharides, such as starch and glycogen, to yield glucose and maltose. It is the major form of amylase found in humans and other mammals. It is also present in seeds containing starch as a food reserve, and is secreted by many microorganisms.

One alpha amylase unit (AAU) of bacterial alpha-amylase activity is the amount of enzyme required to hydrolyze 10 mg of starch per minute from 5% dry substance soluble Lintner starch solution containing 31.2 mM calcium chloride, at 60° C. and 6.0 pH buffered with 30 mM sodium acetate. As understood by those in the art, the quantity of alpha amylase used in the methods of the present invention will depend in part on the enzymatic activity of the particular alpha amylase employed. In some aspects of the methods provided herein, a high concentration of alpha amylase is used to liquefy starch present in a ground or fractionated grain, such as any of the ground or fractionated grains described above. In this context, given the variability in the enzymatic activities of alpha amylases derived from many species of plants, fungus, bacteria, and yeasts, a “high concentration” of alpha amylase means the alpha amylase used for the liquefaction reaction is present at a concentration of between about 1-50 AAU/gds (gram dissolved solids), although in some aspects the alpha amylase is added in an amount between about 2 to 20 AAU/gds, between about 5 to 20 AAU/gds, between about 10-20 AAU/gds or between about 15 to 20 AAU/gds. For example, generally an amount of between about 2 to 10 AAU/gds of SPEZYME® XTRA or SPEZYME® Alpha or SPEZYME® RSL. (Genencor-Danisco) is added per gram of ds.

In some aspects, the high concentration of alpha amylase used for the liquefaction reaction in any of the methods described herein can be any of about 1 AAU/gds, 2 AAU/gds, 3 AAU/gds, 4 AAU/gds, 5 AAU/gds, 6 AAU/gds, 7 AAU/gds, 8 AAU/gds, 9 AAU/gds, or 10 AAU/gds, inclusive, including any values in between these concentrations. In some aspects, the high concentration of alpha amylase used for the liquefaction reaction is greater than about 10 AAU/gds, including concentrations greater than about 12 AAU/gds, about 14 AAU/gds, about 16 AAU/gds, about 18 AAU/gds, or about 20 AAU/gds, inclusive, including any concentrations in between these values. In other aspects, the high concentration of alpha amylase used for the liquefaction reaction is between about 3-9 AAU/gds, 4-8 AAU/gds, or 5-7 AAU/gds, inclusive. In still other aspects, the high concentration of alpha amylase used for the liquefaction reaction is between about 3-10 AAU/gds, 4-10 AAU/gds, 5-10 AAU/gds, 6-10 AAU/gds, 7-10 AAU/gds, 8-10 AAU/gds, or 9-10 AAU/gds, inclusive. In a further aspect, the high concentration of alpha amylase used for the liquefaction reaction is between about 3-9 AAU/gds, 3-8 AAU/gds, 3-7 AAU/gds, 3-6 AAU/gds, 3-5 AAU/gds, or 3-4 AAU/gds, inclusive.

Suitable alpha amylases may be naturally occurring as well as recombinant and mutant alpha amylases. In some aspects of the methods described herein, the alpha amylase is a thermostable bacterial alpha amylase or an acid fungal alpha amylase. Exemplary alpha amylases suitable for use in the methods describe herein include, but are not limited to, an alpha amylase derived from a Bacillus species (such as, for example, alpha amylases derived from B. subtilis, B. stearothermophilus, B. lentus, B. licheniformis, B. coagulans, or B. amyloliquefaciens; see U.S. Pat. Nos. 5,763,385; 5,824,532; 5,958,739; 6,008,026 and 6,361,809, the contents of which are incorporated by reference herein in their entireties). Additional exemplary alpha amylases include those expressed by the American Type Culture Collection (ATCC) strains ATCC 39709; ATCC 11945; ATCC 6598; ATCC 6634; ATCC 8480; ATCC 9945A and NCIB 8059. Commercially available alpha amylases contemplated for use in the methods of the invention include, but are not limited to, SPEZYME® AA; SPEZYME® FRED; SPEZYME® Alpha, SPEZYME® XTRA GZYME™ G997 SPEZYME® RSL (Genencor-Danisco), TERMAMYL™ 120-L, LC, Fuelzyme, Liquozyme SC and Liquozyme SUPRA (from Novozymes).

In some aspects, the alpha amylase employed in any of the methods disclosed herein is “Amy E”, the production and purification of which are described in U.S. Patent Publication Nos.: US2009/0305935-A 1 and US2009/0305360-A 1, the disclosures of which are hereby incorporated by reference in their entireties with respect to teachings related to Amy E.

Glucoamylases

In some aspects, the methods provided herein use at least one glucoamylase for the saccharification of soluble low DE dextrins. Glucoamylases (EC.3.2.1.3; also known as amyloglucosidase, glucoamylase, or alpha-1, 4-D-glucan glucohydrolase) are exo-acting enzymes which release glucosyl residues from the non-reducing ends of amylose and amylopectin molecules. These enzymes also hydrolyze alpha-1,6 and alpha-1,3 linkages although at much slower rates than alpha 1,4 linkages. Glucoamylase can also hydrolyze both linear and branched glucosidic linkages of starch, amylose and amylopectin.

One Glucoamylase Unit (GAU) is the amount of enzyme that liberates one gram of reducing sugars calculated as glucose from a 2.5% dry substance soluble Lintner starch substrate per hour at 60° C. and 4.3 pH buffered with 20 mM sodium acetate. As understood by those in the art, the quantity of glucoamylase used in the methods of the present invention will depend in part on the enzymatic activity of the particular alpha amylase employed. In some aspects, the concentration of glucoamylase used for the saccharification reaction in any of the methods described herein can be any of about 0.01-0.2 GAU/gds, inclusive. In other aspects, the concentration of glucoamylase is between about 0.05-0.15 GAU/gds, inclusive, or between about 0.75-0.1 GAU/gds, inclusive. In still other aspects, the concentration of glucoamylase is between about 0.01-0.2 GAU/gds, 0.03-0.2 GAU/gds, 0.05-0.2 GAU/gds, 0.07-0.2 GAU/gds, 0.09-0.2 GAU/gds, 0.11-0.2 GAU/gds, 0.13-0.2 GAU/gds, 0.15-0.2 GAU/gds, 0.17-0.2 GAU/gds, or 0.19-0.2 GAU/gds, inclusive. In a further aspect, the concentration of glucoamylase is between about 0.01-1.8 GAU/gds, 0.01-1.6 GAU/gds, 0.01-1.4 GAU/gds, 0.01-1.2 GAU/gds, 0.01-1.0 GAU/gds, 0.01-0.8 GAU/gds, 0.01-0.6 GAU/gds, 0.01-0.4 GAU/gds, or 0.01-0.2 GAU/gds, inclusive. In another aspect, the concentration of glucoamylase is any of about 0.025 GAU/gds, about 0.05 GAU/gds, about 0.075 GAU/gds, about 0.1 GAU/gds, or about 0.2 GAU/gds, inclusive, including any concentrations in between these values.

Suitable glucoamylases may be naturally occurring (for example, a glucoamylase derived from bacteria, plants, or fungi) as well as recombinant and mutant glucoamylases. Exemplary glucoamylases suitable for use in the methods described herein include, but are not limited to, glucoamylases secreted from fungi of the genera Aspergillus niger, Aspergillus awamori, Rhizopus niveus, Rhizopus oryzae, Mucor miehe, Humicola grisea, Aspergillus shirousami and Humicola (Thermomyces) laniginosa (see, Boel et al., 1984, EMBO J., 3:1097-1102; International Patent Application Publication Nos. WO 92/00381 and WO 00/04136; Chen et al., 1996, Prot. Eng. 9:499-505; Taylor et al., 1978, Carbohydrate Res., 61:301-308 and Jensen et al., 1988, Can. J. Microbiol. 34:218-223, the disclosures of which are incorporated herein by reference). Other exemplary fungal glucoamylases include those from Trichoderma reesei, Humicola (see U.S. Pat. No. 4,618,579, the disclosure of which is incorporated herein by reference), Humiocla glucoamylase expressed in Trichoderma (see U.S. Pat. No. 7,303,899, the disclosure of which is incorporated herein by reference), Talaromyces species such as Talaromyces emersonii (see International Patent Application No.: WO99/28448, the disclosure of which is incorporated herein by reference), Talaromyces leycettanus (see U.S. Pat. No.: RE32153, the disclosure of which is incorporated herein by reference), Talaromyces dupanti and thermophilus (see U.S. Pat. No. 4,587,215, the disclosure of which is incorporated herein by reference), Cladosporium resinae (formerly known as Harmoconis resinae, see U.S. Pat. No. 4,318,927, the disclosure of which is incorporated herein by reference) and the thermophilus fungus Thermomyces lanuginosus (formerly known as Humicola lanuginose (see Rao et al., 1981, Biochem. J, 193:379-387, the disclosure of which is incorporated herein by reference).

Commercial glucoamylases from a variety of fungal sources suitable for use in the methods of the present invention include, for example, Distillase® L-400, FERMENZYME® L-400, G Zyme™ 480 Ethanol, GC 147, DISTILLASE®SSF from Genencor-Danisco and Spirizyme™ Fuel, Spirizyme™ Plus, Spirizyme™ Plus Tech and Spirizyme™ Ultra from Novozymes.

In some aspects, the glucoamylase employed in any of the methods disclosed herein is “Humicola-Glucoamylase (H-GA)”, the recombinant expression of which in a Trichoderma host is described in U.S. Pat. No. 7,303,899, the disclosure of which is hereby incorporated by reference herein. In other aspects, a Trichoderma host expresses a heterologous polynucleotide which encodes a glucoamylase derived from a Humicola grisea strain, particularly a strain of Humicola grisea var. thermoidea.

Other Carbohydrate and Grain-Degrading Enzymes

In one aspect of any of the methods provided herein, further enzymes may be used to degrade starch and/or other components (such as lipids and proteins) of any of the ground or fractionated grains described above. Exemplary enzymes suitable for such use include, but are not limited to, other amylases (such as beta amylases, AmyE alpha amylase or a variant thereof or isoamylases), beta-galactosidases, catalases, laccases, cellulases, endoglycosidases, endo-beta-1,4-laccases, other glucosidases, glucose isomerases, glycosyltransferases, lipases, phospholipases, lipooxygenases, beta-laccases, endo-beta-1, 3(4)-laccases, cutinases, peroxidases, pectinases, reductases, oxidases, decarboxylases, phenoloxidases, ligninases, pullulanases, arabinanases, hemicellulases, mannanases, xylolaccases, xylanases, pectin acetyl esterases, rhamnogalacturonan acetyl esterases, proteases, peptidases, proteinases, polygalacturonases, rhamnogalacturonases, galactanases, pectin lyases, transglutaminases, pectin methylesterases, cellobiohydrolases and/or transglutaminases.

### Grain-Derived Endogenous Enzymes

In some aspects of the methods disclosed herein, endogenous enzymes present in one or more whole ground or fractionated grains, such as any of the grains described herein, can be used to produce a fermentable sugar feedstock. Without being bound to theory, certain unrefined grains (for example, ground corn or milo) or fractionated components thereof (such as an endosperm fraction) can contain one or more endogenous enzyme(s) capable of degrading starch into less complex saccharides possessing a distinct DP2 profile (such as a DP2 profile enriched in kojibiose and nigerose). When used in combination with a high concentration of an alpha amylase and a glucoamylase (such as any alpha amylase or glucoamylase described above), the starch-degrading activity of the endogenous enzyme fraction can be used to solubilize greater than about 90% (such as greater than about 91%, 92%, 93%, 94%, 95%, 96%, 97%, or 98%) of the starch in the grains to fermentable sugars.

Accordingly, in some aspects of the methods described herein, the endogenous enzyme activity can be present in a whole ground or a fractionated grain and, when used in combination with a high concentration of an alpha amylase and a glucoamylase at temperatures at or below the initial gelatinization temperature of the starch in the grain, can degrade the starch in the whole ground or fractionated grain into a fermentable sugar feedstock containing the disaccharides kojibiose and nigerose. In some aspects of the methods described herein, the endogenous enzyme-containing whole ground or fractionated grain can be ground corn, corn endosperm, whole ground milo, or whole ground rice

In other aspects of the methods described herein, the whole ground or fractionated grain lacks the endogenous enzyme activity described above or the starch source used for production of a fermentable sugar feedstock is a refined starch, such as corn starch, wheat starch, or rice starch, which also lacks the endogenous enzyme activity described above. In this case, a fraction containing the endogenous enzymes can be isolated from a whole ground or fractionated grain which does contain the endogenous enzyme activity for use in the production of a fermentable sugar feedstock that is enriched in the disaccharides kojibiose and nigerose. In some aspects, an isolated fraction containing the endogenous enzyme activity can be obtained by centrifuging an aqueous slurry of a grain that contains the endogenous enzymes (such as an aqueous slurry of ground corn, corn endosperm, whole ground milo, whole ground rice, or whole ground wheat) and isolating the supernatant. For example, an aqueous slurry can be prepared by mixing any of the endogenous enzyme-containing grains described above with DI water in order to produce a slurry with any of about 10%, about 15%, about 20%, about 25%, about 30%, about 35%, about 40%, about 45%, or about 50% dry solids, inclusive, including any percentages in between these values. The slurry can then be incubated at room temperature for any of about 30 min, about 60 min, about 90 min, about 120, about 150 min, about 180 min, about 210 min, about 240 min, about 270 min, or about 300 min, inclusive, including any times in between these values. Following incubation, the slurry can centrifuged to remove heavy solids and the resulting supernatant (extract) will contain the endogenous enzyme fraction. Other methods for extraction of endogenous proteins such as enzymes from plant material such as grains may also be used. These methods are numerous and well known in the art (see, for example, U.S. Pat. No. 6,740,740, the contents of which are incorporated by reference herein in its entirety).

In other aspects of the methods provided herein, it may be advantageous to deactivate the endogenous enzyme activity present in certain grains (such as the grains described above), so that the fermentable sugar feedstock produced from these grains is not enriched in DP2 sugars, such as kojibiose and nigerose. In one aspect, the endogenous enzyme activity described above can be deactivated by temporarily lowering the pH of an aqueous slurry of the grain containing the endogenous enzyme activity. In some aspects, the pH is lowered to any of about pH 1, 1.5, 2, 2.5, 3, 3.5, or 4 to inactivate the endogenous enzyme activity. In other aspects, the slurry is exposed to lowered pH for any of about 30 min, about 60 min, about 90 min, about 120, about 150 min, or about 180 min, inclusive, including any times in between these numbers. In yet other aspects, the slurry is exposed to lowered pH at a temperature of about 37° C., 40° C., 45° C., 50° C., 55° C., 60° C., 65° C., 70° C., 75° C., or 80° C., inclusive, including any temperatures in between these values.

### Methods of the Invention

Provided herein are methods for making a fermentable sugar feedstock by treating an aqueous slurry of ground or fractionated grain (such as any of the ground or fractionated grains described above) with a high concentration of a starch solubilizing alpha amylase and a glucoamylase. The treatment is conducted at a temperature at or below the initial gelatinization temperature of the starch in the grain. Additionally, the fermentable sugar feedstocks produced by the methods disclosed herein can possess a higher concentration of DP-2 saccharides in comparison to sugar feedstocks that are made by other methods.

Also provided herein are methods for the production of a fermentable sugar feedstock by treating a refined granular starch or a whole or fractionated grain that lacks an endogenous enzyme activity with a high concentration of a starch solubilizing alpha amylase, a glucoamylase, and an enzyme-containing extract isolated from a grain. Additionally, the fermentable sugar feedstocks produced by the methods disclosed herein can possess a higher concentration of DP-2 saccharides in comparison to sugar feedstocks made by other methods In some aspects, the enzyme-containing extract is derived from whole ground corn, corn endosperm, whole ground milo, whole ground rice, or any combinations thereof using any of the derivation methods disclosed herein. In another aspect, the refined granular starch is corn starch, rice starch, or wheat starch.

In some aspects of the methods provided herein, treatment of the slurry with a high concentration of alpha amylase (as further detailed below) and a glucoamylase is conducted at a temperature at or below the initial gelatinization temperature of the starch in the grain. The initial gelatinization temperature of starch depends upon many variable such as the grain type and the conditions in which it was grown (e.g. water availability), the amount of water present in the aqueous slurry, the pH, and the types and amount of other molecules present in the grains (such as lipids and protein). Some types of unmodified native starches start swelling at 55° C., other types at 85° C. (Belitz et al., Food Chemistry, 3^{rd} ed., (2004, Springer), pg. 318-23). The initial gelatinization temperature can also depend on the degree of cross-linking of amylopectin (one of the two components of starch, the other being amylose) which is determined in large part by the action of one or more endogenous starch synthase genes (see, e.g., U.S. Patent Application Publication No.: 2008/0201807). Consequently, in some aspects, the treatment is conducted at a temperature between about 0° C. to about 40° C. (such as between about 0° C. to about 30° C.) below the initial gelatinization temperature of the starch present in a particular grain used in any of the methods disclosed herein. In other aspects, the treatment is conducted at a temperature between about 0 to 5° C., between about 2 to 7° C., between about 4 to 9° C., between about 6 to 11° C., between about 8 to 13° C., between about 10 to 15° C., between about 12 to 17° C., between about 13 to 19° C., between about 15 to 21° C., between about 17 to 23° C., between about 19 to 25° C., between about 21 to 27° C., between about 23 to 29° C., between about 25 to 31° C., between about 27 to 33° C., between about 29 to 35° C., between about 31 to 37° C., between about 33 to 39° C. or between about 35 to 40° C., inclusive, below the initial gelatinization temperature of the starch present in a particular grain used in any of the methods disclosed herein. In some aspects, the treatment is conducted at a temperature between about 55-65° C., inclusive. In other aspects, the treatment is conducted at a temperature between about 57-65° C., 59-65° C., 61-65° C., or 63-65C ° C., inclusive. In yet other aspects, the treatment is conducted at a temperature between about 55-63° C., 55-61° C., 55-59° C., or 55-57° C., inclusive. In other aspects, the treatment is conducted at a temperature at least about 55° C., at least about 57° C., at least about 59° C., at least about 61° C., at least about 63° C., or at least about 65° C. In still further aspects, the treatment is conducted at a temperature no greater than about 65° C., about 63° C., about 61° C., about 59° C., about 57° C., or about 55° C.

In other aspects of the methods provided herein, treatment of the slurry with a high concentration of alpha amylase (as further detailed below) and a glucoamylase can be conducted for between about 12-60 hours. In some aspects, the treatment of the slurry is conducted for between about 12-55 hours, 12-50 hours, 12-45 hours, 12-40 hours, 12-35 hours, 12-30 hours, 12-25 hours, 12-20 hours, or 12-15 hours. In still further aspects, the treatment of the slurry is conducted for between about 15-60 hours, 20-60 hours, 25-60 hours, 30-60 hours, 35-60 hours, 40-60 hours, 45-60 hours, 50-60 hours, or 55-60 hours. In other aspects, the treatment of the slurry is conducted for 15-55, 20-50, 25-45, or 30-40 hours. In another aspect, the treatment of the slurry is conducted for any of about 2 hours, about 4 hours, about 6 hours, about 12 hours, about 24 hours, or about 48 hours, inclusive, including any time in between these numbers.

In some aspects the starch solubilizing alpha amylase can be any of the alpha amylases disclosed above (including an acid fungal alpha amylase, an AmyE amylase, or an AmyE variant amylase) and the high concentration used in the methods described herein can be any of about 1 AAU/gds, 2 AAU/gds, 3 AAU/gds, 4 AAU/gds, 5 AAU/gds, 6 AAU/gds, 7 AAU/gds, 8 AAU/gds, 9 AAU/gds, or 10 AAU/gds, inclusive, including any values in between these concentrations. In some aspects, the high concentration of alpha amylase is greater than about 10 AAU/gds, including concentrations greater than about 12 AAU/gds, about 14 AAU/gds, about 16 AAU/gds, about 18 AAU/gds, or about 20 AAU/gds, inclusive, including any concentrations in between these values. In other aspects, the high concentration of alpha amylase used for the liquefaction reaction is between about 3-9 AAU/gds, 4-8 AAU/gds, or 5-7 AAU/gds, inclusive. In still other aspects, the high concentration of alpha amylase used for the liquefaction reaction is between about 3-10 AAU/gds, 4-10 AAU/gds, 5-10 AAU/gds, 6-10 AAU/gds, 7-10 AAU/gds, 8-10 AAU/gds, or 9-10 AAU/gds, inclusive. In a further aspect, the high concentration of alpha amylase used for the liquefaction reaction is between about 3-9 AAU/gds, 3-8 AAU/gds, 3-7 AAU/gds, 3-6 AAU/gds, 3-5 AAU/gds, or 3-4 AAU/gds, inclusive.

In other aspects, the glucoamylase can be any of the glucoamylases described above and the concentration of glucoamylase used for the methods described herein can be any of about 0.01-0.2 GAU/gds, inclusive. In other aspects, the concentration of glucoamylase is between about 0.05-0.15 GAU/gds, inclusive, or between about 0.75-0.1 GAU/gds, inclusive. In still other aspects, the concentration of glucoamylase is between about 0.01-0.2 GAU/gds, 0.03-0.2 GAU/gds, 0.05-0.2 GAU/gds, 0.07-0.2 GAU/gds, 0.09-0.2 GAU/gds, 0.11-0.2 GAU/gds, 0.13-0.2 GAU/gds, 0.15-0.2 GAU/gds, 0.17-0.2 GAU/gds, or 0.19-0.2 GAU/gds, inclusive. In a further aspect, the concentration of glucoamylase is between about 0.01-1.8 GAU/gds, 0.01-1.6 GAU/gds, 0.01-1.4 GAU/gds, 0.01-1.2 GAU/gds, 0.01-1.0 GAU/gds, 0.01-0.8 GAU/gds, 0.01-0.6 GAU/gds, 0.01-0.4 GAU/gds, or 0.01-0.2 GAU/gds, inclusive. In another aspect, the concentration of glucoamylase is any of about 0.025 GAU/gds, about 0.05 GAU/gds, about 0.075 GAU/gds, about 0.1 GAU/gds, or about 0.2 GAU/gds, inclusive, including any concentrations in between these values.

In other aspects, the methods described herein may further include the additional step of adding one or more other enzymes used to degrade starch and/or other components (such as lipids and proteins) of any of the ground or fractionated grains described above. In one aspect, the methods described herein further comprise the addition of one or more other amylases (such as beta amylases, AmyE alpha amylase or a variant thereof or isoamylases), beta-galactosidases, catalases, laccases, cellulases, endoglycosidases, endo-beta-1,4-laccases, other glucosidases, glucose isomerases, glycosyltransferases, lipases, phospholipases, lipooxygenases, beta-laccases, endo-beta-1, 3(4)-laccases, cutinases, peroxidases, pectinases, reductases, oxidases, decarboxylases, phenoloxidases, ligninases, pullulanases, arabinanases, hemicellulases, mannanases, xylolaccases, xylanases, pectin acetyl esterases, rhamnogalacturonan acetyl esterases, proteases, peptidases, proteinases, polygalacturonases, rhamnogalacturonases, galactanases, pectin lyases, transglutaminases, pectin methylesterases, cellobiohydrolases and/or transglutaminases.

In a further aspect of any of the methods provided herein, greater than 80% of the ground or fractionated grain present in the aqueous slurry (such as an aqueous slurry of any of the ground or fractionated grains described above) is solubilized by treatment with a high concentration of a starch solubilizing alpha amylase and a glucoamylase and at a temperature at or below the initial gelatinization temperature of the starch in the grain. In other aspects, at least about 82%, 84%, 86%, 88%, 90%, 91%, 92%, 93%, 94%, 95%, 96%, 97%, 98%, 99%, or 100% of the ground or fractionated grain is solubilized. In another aspect of the methods provided herein, the solubilized starch can be greater than about 80% fermentable sugars (such as greater than about 82%, 84%, 86%, 88%, 90%, 91%, 92%, 93%, 94%, 95%, 96%, 97%, 98%, 99%, or 100% fermentable sugars).

Depending on the treatment conditions used in the methods described herein (such as for example, length of treatment, concentration of alpha amylase and glucoamylase, temperature of the treatment, or any other treatment variable disclosed herein), the fermentable sugar feedstocks will comprise primarily DP1 and DP2 sugars. In some aspects, the fermentable sugar feedstock comprises at least about 60% DP1 sugars (for example, glucose). In another aspect, the fermentable sugar feedstocks can comprise at least about 60%, 65%, 70%, 75%, 80%, 82%, 84%, 86%, 88%, 90%, 91%, 92%, 93%, 94%, or 95% DP1 sugars, inclusive, including any percentages in between these values. In a further aspect, the fermentable sugar feedstock can comprise no more than about 30% DP2 sugars (for example, maltose or isomaltose). In another aspect, the fermentable sugar feedstock can comprise no more than about 27%, 25%, 20%, 15%, 14%, 13%, 12%, 11%, 10, 9%, 8%, 7%, 6%, 5%, 4%, 3%, 2%, or 1%, inclusive, including any percentage in between these values, DP2 sugars.

The fermentable sugar feedstock produced using any of the methods disclosed herein can contain a higher concentration of DP2 saccharides in comparison to fermentable sugar feedstocks produced by methods wherein ground or fractionated grain present in the aqueous slurry are not solubilized by treatment with a high concentration of a starch solubilizing alpha amylase and a glucoamylase and at a temperature at or below the initial gelatinization temperature of the starch in the grain. In one aspect, the feedstocks produced using the methods described herein can be rich in DP2 saccharides, such as kojibiose and nigerose. Kojibiose (2R,3S,4R,5R)-3,4,5,6-tetrahydroxy-2-[(2R,3R,4S,5S,6R)-3,4,5-trihydroxy-6-(hydroxymethyl)oxan-2-yl]oxyhexanal) is a disaccharide and is commonly observed as a byproduct of the caramelization of glucose. Nigerose ((2R,3S,4S,5R,6R)-2-(Hydroxymethyl)-6-[(3R,4S,5R,6R)-2,3,5-trihydroxy-6-(hydroxymethyl)oxan-4-yl]oxyoxane-3,4,5-triol), also a byproduct of the caramelization of glucose, is a disaccharide made of two glucose residues, connected with an alpha 1-3 link. In some aspects, the DP2 saccharide concentration within feedstocks produced by the methods disclosed herein can comprise any of about 0.1% DP2 g/100 gds, 0.5% DP2 g/100 gds, 1% DP2 g/100 gds, 2% DP2 g/100 gds, 3% DP2 g/100 gds, 4% DP2 g/100 gds, 5% DP2 g/100 gds, 6% DP2 g/100 gds, or 7% DP2 g/100 gds, inclusive, including any percentages in between these values, kojibiose. In other aspects, the DP2 saccharide concentration within feedstocks produced by the methods disclosed herein can comprise any of about 0.1% DP2 g/100 gds, 0.5% DP2 g/100 gds, 1% DP2 g/100 gds, 2% DP2 g/100 gds, 3% DP2 g/100 gds, 4% DP2 g/100 gds, or 5% DP2 g/100 gds, inclusive, including any percentages in between these values, nigerose.

Methods for Making a Fermentable Sugar Feedstock Having a Reduced Concentration of DP2 Saccharides

In some aspects, it may be desirable to produce a fermentable sugar feedstock that is not enriched in the DP2 saccharides kojibios and nigerose. In these aspects, the following steps can be used: (a) inactivating endogenous enzyme activity in a whole or fractionated grain and (b) treating the whole or fractionated grain with a high concentration of alpha amylase and a glucoamylase. The treatment is at a temperature at or below the initial gelatinization temperature of the starch in the grain. In one aspect, the fermentable sugar feedstock can have a decreased concentration of DP-2 saccharides in comparison to fermentable sugar feedstocks that are not made by inactivating endogenous enzyme activity in a whole or fractionated grain. In some aspects, the whole or fractionated grain is whole crown corn or corn endosperm. In some aspects, fermentable sugar feedstocks made using methods wherein endogenous enzyme activity is inactivated contain 0% DP2 g/100 gds or near 0% DP2 g/100 gds concentrations of kojibiose and/or nigerose.

Endogenous enzyme activity in the a whole or fractionated grain can be inactivated by using any of the methods disclosed herein, including, but not limited to, exposure of an aqueous slurry containing the whole or fractionated grain to low pH. In some aspects, the pH is lowered to any of about pH 1, 1.5, 2, 2.5, 3, 3.5, or 4 to inactivate the endogenous enzyme activity. In other aspects, the slurry is exposed to lowered pH for any of about 30 min, about 60 min, about 90 min, about 120, about 150 min, or about 180 min, inclusive, including any times in between these numbers. In yet other aspects, the slurry is exposed to lowered pH at a temperature of about 37° C., 40° C., 45° C., 50° C., 55° C., 60° C., 65° C., 70° C., 75° C., or 80° C., inclusive, including any temperatures in between these values.

### Use of Fermentable Sugar Feedstocks for the Production of One or More Industrial Products

The fermentable sugar feedstocks produced by any of the methods disclosed herein may be further purified and/or converted to useful sugar products (such as sugar syrups). In addition, the sugars may be used as a fermentation feedstock in a fermentation process, such as a microbial fermentation process, for producing one or more end-products, such as an alcohol (e.g., ethanol and butanol), an organic acid (e.g., succinic acid and lactic acid), a sugar alcohol (a.k.a. a “polyol”; e.g., glycerol), ascorbic acid intermediates (e.g., gluconate, 2-keto-D-gluconate, 2,5-diketo-D-gluconate, and 2-keto-L-gulonic acid), amino acids (e.g., lysine), proteins (e.g., antibodies and fragments thereof), the production of enzymes, or the production of biofuels, for example ethanol and butanol.

The microorganism used in fermentations will depend on the desired end-product. For example, if ethanol is the desired end product, yeast will be used as the fermenting organism. In some aspects, the ethanol-producing microorganism is a yeast and specifically Saccharomyces such as strains of S. cerevisiae (see, e.g. U.S. Pat. No. 4,316,956). A variety of S. cerevisiae are commercially available and these include, but are not limited to, FALI (Fleischmann's Yeast), SUPERSTART (Alltech), FERMIOL (DSM Specialties), RED STAR (Lesaffre) and Angel alcohol yeast (Angel Yeast Company, China). The amount of starter yeast employed, is an amount effective to produce a commercially significant amount of ethanol in a suitable amount of time, (e.g. to produce at least 10% ethanol from a substrate having between 25-40% DS in less than 72 hours). Yeast cells are generally supplied in amounts of about 104 to about 1012, and preferably from about 107 to about 1010 viable yeast count per mL of fermentation broth. After yeast is added to the mash, it can be subjected to fermentation for about 24-96 hours, e.g., 35-60 hours. The temperature can be about 26-34° C., for example, about 32° C., and the pH can be from pH 3-6, preferably around pH 4-5. The fermentation may include, in addition to a fermenting microorganisms (e.g. yeast), nutrients, and additional enzymes, including phytases and/or other enzymes to enhance the rate and/or increase the solubilization of granular starch. The use of yeast in fermentation is well known and reference is made to The Alcohol Textbook, Jacques et al., Eds. (1999, Nottingham University Press, UK).

In other aspects, the fermenting organism can be a species of yeast other than S. cerevisiae such as, but not limited to, a Pichia spp., a Candida spp., a Hansenula spp., a Kluyveromyces spp., a Kluyveromyces spp., or a Schizosaccharomyces spp. In still other aspects, the fermenting organism can be a species of bacterium including, but not limited to, an Arthrobacter spp., an Escherichia spp., a Zymomonas spp., a Brevibacterium spp., a Clostridium spp., an Aerococcus spp., a Bacillus spp., a Carbobacterium spp., a Corynebacterium spp., an Enterococcus spp., an Erysipelothrix spp., a Gemella spp., a Geobacillus spp., a Globicatella spp., a Lactobacillus spp., a Lactococcus spp., a Leuconostoc spp., a Pediococcus spp., a Streptococcus spp., a Tetragenococcus spp., an Actinobacillus spp., or a Vagococcus spp., In other aspects, the fermenting organism can be a fungus such as, but not limited to, a Rhizopus spp.

Optionally, following fermentation, alcohol (e.g., ethanol or butanol) may be extracted by, for example, distillation and optionally followed by one or more process steps. In some aspects, the yield of ethanol produced by the methods provided herein is at least 8%, at least 10%, at least 12%, at least 14%, at least 15%, at least 16%, at least 17% and at least 18% (v/v) and at least 23% v/v. The ethanol obtained according to the process provided herein may be used as, for example, fuel ethanol, drinking ethanol, i.e., potable neutral spirits, or industrial ethanol. The butanol obtained according to the processes provided herein may be used as, for example, fuel additives or industrial butanol.

If lactic acid is the desired end product, a Lactobacillus species of bacteria such as, but not limited to, L. lactis or L. rhammosus can be used as the fermenting organism. In some aspects, the lactic acid producing microorganism includes natural and/or selected microorganisms or microorganisms produced by adaptation or mutated to produce lactic acid. Producer organisms include lactic acid bacteria, such as those of the genera Aerococcus, Bacillus, Carbobacterium, Enterococcus, Erysipelothrix, Gemella, Globicatella, Lactobacillus, Lactococcus, Leuconostoc, Pediococcus, Streptococcus, Tetragenococcus and Vagococcus. For example, other bacteria of the genus Lactobacillus which may be substituted include, but are not limited to, L. heiveticus, L. delbrueckii, L. casei, L, acidophilus, L. amylovorus, L. leichmanii or L. bulgaricus. L. amylovorus and L. pentosus. The amount of producer organism employed is an amount effective to produce a commercially significant amount of lactic acid in a suitable amount of time. In some aspects, the fermentation can further comprise the step of adding additional glucoamylase (e.g. 0.1 GAU/gds) to the medium.

If succinic acid is the desired end product, an Actinobacillus species of bacterium such as, but not limited to A. succinogens 130Z (ATCC 55618), can be used as the fermenting organism. In some aspects, the succinic acid producing microorganism includes natural and/or selected microorganisms or microorganisms produced by adaptation or mutated to produce succinic acid. The amount of producer organism employed is an amount effective to produce a commercially significant amount of succinic acid in a suitable amount of time.

If an enzyme is the desired end product, one or more species of bacterium such as a Bacillus spp. (e.g. Bacillus subtilis), Trichoderma spp. (e.g. Trichoderma reesei), or Escherichia spp. (e.g. Escherichia coli) can be used as the fermenting organism. In some aspects, the enzymes can be one or more enzymes used for the digestion or the hydrolysis of lignocellulosic biomass to simpler and less highly branched saccharides (e.g. saccharides having a DP of 4 or less, such as any of DP3, DP2, and/or DP1). Non-limiting examples of these simpler and less highly branched oligosaccharides include glucose, xylose, maltose, fructose, xylose, arabinose and/or mannose. The enzymes may also be used for the hydrolysis of starch contained in one or more agricultural products including, but not limited to, corn, barley, wheat, rye, sorghum, cassava, rice, potato, sweet potato, beet, cane (such as sugar cane), or molasses. In other aspects, the enzymes can be used as additives for the processing of textiles or as components of detergent compositions, such as those used for removing stains from fabrics or for household cleaning compositions. The enzymes may also be used as additives for food (such as an additive used for baking), as a component of an animal feed, as an additive used for the production of pulp or paper, as a catalyst for the production of a polymer or plastic, or as a pharmaceutical or in the production of a pharmaceutical (such as an antimicrobial). Non-limiting examples of such enzymes include glucoamylases, amylases (such as α-amylases, β-amylases, and/or isoamylases), pullulanases, cellulases, xylanases, hemicellulases, proteases, phytases, lipases, esterases, cutinases, pectinases, oxidases, catalases, transferases (including amino transferases), glucose isomerases, glucosidases (e.g., α-glucosidases), isomerases (e.g., glucose isomerases, or enzymes involved in the anabolism of one or more amino acids (such as glutamic acid or lysine).

In further aspects, use of appropriate fermenting microorganisms, as is known in the art, can result in fermentation end product including, e.g., glycerol, 1,3-propanediol, gluconate, 2-keto-D-gluconate, 2,5-diketo-D-gluconate, 2-keto-L-gulonic acid, lactic acid, amino acids, gluconic acid, succinic acid, citric acid, monosodium glutamate, sodium gluconate, calcium gluconate, potassium gluconate, glucono delta lactone, sodium erythorbate, itaconic acid, ketones, amino acids, glutamic acid (sodium monoglutamate), penicillin, tetracyclin, enzymes, vitamins, hormones and derivatives thereof. More specifically when glycerol or 1,3-propanediol are the desired end-products E. coli may be used; and when 2-ketoD-gluconate, 2,5-diketo-D-gluconate, and 2-keto-L-gulonic acid are the desired end products, Pantoea citrea may be used as the fermenting microorganism. The above enumerated list are only examples and one skilled in the art will be aware of a number of fermenting microorganisms that may be used to obtain a desired end product.

In other aspects, the fermentable sugar feedstocks produced by any of the methods disclosed herein can be used for the industrial production of an enzyme by one or more fermenting microorganisms. Depending on the type of enzyme to be produced, the fermenting microorganism can be a bacterium, such as, but not limited to, E. coli. In other aspects, the purified enzyme produced using the feedstocks provided herein can be further used for such applications as the processing of grain, as an additive or in the preparation of food, as an additive or in the preparation of animal feed, as an ingredient in a detergent or cleaning agent, in the processing of textiles, as a pharmaceutical, or in the processing of pulp for the manufacture of paper.

The invention can be further understood by reference to the following examples, which are provided by way of illustration and are not meant to be limiting.

### EXAMPLES

### Example 1

### The Effect of Grain Endogenous Starch Hydrolyzing Activity on the Solubilization of Granular Starch and Production of Fermentable Sugars at Temperatures Below that of the Gelatinization Temperature of the Grain

In this Example, alpha amylase is used in combination with an endogenous starch hydrolyzing enzyme fraction derived from unprocessed grain to synergistically produce fermentable sugars having a DP of 1 or 2 at temperatures below the initial gelatinization temperature of the starch in the grain.

### Materials and Methods

Alpha-amylase Activity (AAU):

One AAU of bacterial alpha-amylase activity is the amount of enzyme required to hydrolyze 10 mg of starch per min from 5% dry substance soluble Lintner starch solution containing 31.2 mM calcium chloride, at 60° C. and 6.0 pH buffered with 30 mM sodium acetate.

Sugar composition determined by HPLC:

The composition of the reaction products of oligosaccharides was measured by high pressure liquid chromatographic method (Beckman System Gold 32 Karat Fullerton, Calif., USA) equipped with a HPLC column (Rezex 8 u8% H, Monosaccharides), maintained at 50° C. fitted with a refractive index (RI) detector (ERC-7515A, RI Detector from The Anspec Company, Inc.). Dilute sulfuric acid (0.01 N) was used as the mobile phase at a flow rate of 0.6 ml per minute. Twenty microliter of 4.0% solution was injected onto the column. The column separates based on the molecular weight of the saccharides. For example a designation of DP1 is a monosaccahride, such as glucose; a designation of DP2 is a disaccharide, such as maltose; a designation of DP3 is a trisaccharide, such as maltotriose and the designation “DP4+” is an oligosaccharide having a degree of polymerization (DP) of 4 or greater.

Percent solubilization of granular starch:

Solubility testing is done by sampling from the agitated slurry into 2.5 ml micro-centrifuge tubes. The tubes are spun for ˜4 minutes at 13,000 rpm and the refractive index of the supernatant is determined at 30C (RIₛᵤₚ). The total dry substance is determined by taking 1.5-2 ml of the starch slurry into a 2.5 ml spin tube, adding 1 drop of SPEZYME® FRED from a micro disposable-pipete then boiling 10 minutes. The tubes are spun for ˜4 minutes at 13,000 rpm and the refractive index of the supernatant is determined at 30° C. (RIₜₒₜ). The dry substance of the supernatant and the whole sample (total) are determined using appropriate DE tables. Table for converting RIₛᵤₚ to DS is the 42 DE, Table I from the Critical Data Tables of the Corn Refiners Association, Inc. To convert RIₜₒₜ to DS, more than one table can be used and an interpolation between the 32 DE and 42 DE tables employed. First an estimation of the solubilisation is made by dividing the DS from the supernatant by the starting DS*1.05. This estimated solubilization is used for the interpolation between the DS obtained via the 42DE and 32DE table. Solubility is defined as the dry substance of the supernatant divided by the total dry substance times 100. This value is then corrected to compensate for the impact of remaining granular starch.

This experiment was run on three different granular starch substrates: (A) Whole ground corn—150 g passed through 40-mesh sieve and having a moisture content of 14.6%; (B) Corn endosperm (obtained from Valero, Jefferson, Wis.) ground to pass through a 40-mesh sieve and having a moisture content of 14.4%; and (C) Refined corn starch—having a moisture content of 11.8%. Each substrate was weighed and transferred to a screw-capped glass jar to make a final 50 g slurry with water adjusting to 30% DS for whole ground corn and corn endosperm and 20% DS for refined starch.

The pH of the slurry was adjusted to pH 5.5 using 6N hydrochloric acid. SPEZYME® XTRA (from Genencor-Danisco) was added at 8.0 AAU/ds. The temperature was maintained at 60° C. During the incubation, the slurry was gently stirred in a shaking incubator. After time internals of 24 and 48 hours, the Refractive Index method was used to determine % starch solubilized and sugar compositions were determined.

TABLE 1
Solubility, DP, and percentage fermentables and fermentable sugar index obtained
from substrates.

| | | Incubation | | | | | | % | |
|---|---|---|---|---|---|---|---|---|---|
| | | Time | % | % | % | % | % | Fermentables | Fermentable |
| Substrates | Treatments | (Hour) | Solubility | DP1 | DP2 | DP3 | DP4+ | (DP1 + DP2) | Sugar Index |
| Corn | No added | 0 | 5.4 | 20.25 | 3.28 | 6.40 | 41.65 | 23.5 | 0.49 |
| endosperm | enzyme | 24 | 8.7 | 72.09 | 4.49 | 5.18 | 18.24 | 76.6 | 3.27 |
| | | 48 | 8.7 | 69.45 | 6.08 | 5.47 | 18.99 | 75.5 | 3.08 |
| | Spezyme | 24 | 69.5 | 44.44 | 27.88 | 15.2 | 12.48 | 72.3 | 2.61 |
| | Xtra added | 48 | 72.7 | 51.79 | 27.67 | 12.57 | 7.98 | 79.5 | 3.87 |
| Whole | Spezyme | 0 | 8.1 | 15.20 | 33.30 | 8.50 | 42.68 | 48.5 | 0.95 |
| ground | Xtra added | 24 | 67.8 | 55.06 | 23.23 | 10.46 | 11.25 | 78.3 | 3.61 |
| corn | | 48 | 78.6 | 64.35 | 21.06 | 7.57 | 7.02 | 85.4 | 5.85 |
| Refined | Spezyme | 24 | 76.1 | 3.95 | 13.42 | 16.27 | 66.36 | 17.4 | 0.21 |
| corn | Xtra added | 48 | 84.0 | 4.73 | 15.13 | 18.29 | 61.85 | 19.9 | 0.25 |
| starch | | | | | | | | | |

### Results

The data in Table 1 show that the incubation of refined starch (pure starch) with SPEZYME® XTRA, at 60° C. resulted in greater than 80% solubilization of the granular corn starch. At the same time, however, the soluble sugar syrup contained only about 20% fermentable sugars with a low fermentable sugar index of 0.25. In contrast, incubation of whole ground corn or endosperm (without germ and crude fiber) with SPEZYME® XTRA resulted in not only high solubilization of granular starch, but also a significantly high level of fermentable sugars. Without being bound by theory, it is believed that the high level of fermentable sugars obtained suggests the role of endogenous starch hydrolyzing enzymes which are present in whole ground corn or corn endosperm but that absent from refined corn starch. As can be seen from Table 1, the fermentable sugar index of whole ground corn or fractionated corn was increased to greater than 3 compared to 0.25 which was observed using refined starch as substrate during incubation with SPEZYME® XTRA. The granular starch solubilizing effect of SPEZYME® XTRA coupled to the hypothesized fermentable sugar-producing endogenous enzymes in the grain, produced a soluble high fermentable sugar yield using granular starch feed stock.

### Example 2

### Solubilization and Fermentable Sugar Composition of De-Hulled Milo Incubated with Alpha Amylase and Glucoamylase

This study used alpha amylase in combination with glucoamylase (GA) to produce fermentable sugars at temperatures below the initial gelatinization temperature of the starch in the grain.

### Materials and Methods

Glucoamylase Activity Units (GAUs):

One Glucoamylase Unit is the amount of enzyme that liberates one gram of reducing sugars calculated as glucose from a 2.5% dry substance soluble Lintner starch substrate per hour at 60° C. and 4.3 pH buffered with 20 mM sodium acetate.

Whole ground de-hulled Milo (obtained from ICM, Colwich, Kans.) having a moisture content of 13.03% was screened through 30-mesh sieve. An aqueous slurry containing 30% ds was prepared and the pH of the slurry was adjusted to pH 5.5 using 6N hydrochloric acid. SPEZYME® RSL was added at a concentration of 8.0 AAU/ds. To one of the flasks, H-GA was added at a concentration of 0.1 GAU/gds. The temperature was maintained at 60° C. During the incubation, the slurry was gently stirred for uniform mixing. After 48 hours, the Refractive Index method was used to determine percent starch solubilized and sugar composition of the solubilized sugars was determined using HPLC.

TABLE 2
Solubility, DP, and percentage fermentable sugars obtained from de-hulled milo.

| | | Incubation | % | % | % | % | % | % Fermentable |
|---|---|---|---|---|---|---|---|---|
| | Treatments | Time (Hour) | Solubility | DP1 | DP2 | DP3 | DP4+ | sugars (DP1 + DP2) |
| Dehulled | No treatment | 0 | 10.8 | 15.48 | 24.31 | 9.55 | 50.66 | 39.79 |
| Milo | No H-GA | 48 | 77.7 | 72.51 | 15.36 | 5.6 | 6.53 | 87.9 |
| | added | | | | | | | |
| | 0.1 GAU/gds | 48 | 92 | 85.76 | 9.42 | 1.44 | 3.39 | 95.2 |

### Results

Incubation of de-hulled milo with SPEZYME® RSL solubilized greater than 75% of the Milo granular starch in 48 hours at 60° C. The solubilized starch was further hydrolyzed to produce a fermentable sugar syrup containing 87.9% DP1 and DP2. Further addition of 0.1 GAU of H-GA per gram ds resulted in greater than 90% solubilization of the Milo granular starch containing 95% fermentable sugar. This studies shows that alpha amylase in combination with glucoamylase can be used in combination to produce high percentage yields of fermentable sugars at temperatures below that of the initial gelatinization temperature of starch present in de-hulled milo.

### Example 3

### Effect of pH on the Solubilization and Hydrolysis of Corn Endosperm Granular Starch During Incubation with Alpha Amylase

This study examined the effect of pH on the solubilization and hydrolysis of corn endosperm granular starch (30% DS) during incubation with alpha-amylase at a temperature below the gelatinization temperature of starch.

### Materials and Methods

Ground corn endosperm having a moisture content of 14.4% was weighed and transferred to different screw-capped glass jars and water was added to a final ds of 30%. The pH was adjusted to pH 4.5, 5.0, 5.5 and 6.0, respectively. The aqueous slurry was stirred well and the pH was adjusted until the pH was stabilized to the target pH. The jar was pre-warmed for one hour in a 60° C. shaking incubator, followed by addition of SPEZYME® RSL at a concentration of 8.0 AAU/gds. The slurry was then gently stirred for 15 hours. The percent solubilization was then determined as described in Example 1.

TABLE 3
pH optimum for percent solubilization of grain using alpha-amylase
below the initial gelatinization temperature of the grain.

| pH | 4.5 | 5.0 | 5.5 | 6.0 |
|---|---|---|---|---|
| Solubilization | 23.8% | 66.5% | 68.3% | 66.3% |

### Results

The results are shown in Table 3. The experimental results indicate that maximum solubilization of the granular starch occurred at the pH optimum for SPEZYME® RSL which is pH 5.

### Example 4

### Effect of Alpha-Amylase Concentration on the Solubilization and Hydrolysis of Corn Endosperm Granular Starch

The experiment was conducted to study the effect of variable alpha-amylase concentrations on the solubilization of corn endosperm (30% ds) during incubation at pH 5.5 at a temperature below the gelatinization temperature of the starch in the corn endosperm.

### Materials and Methods

SPEZYME® RSL at concentrations of 2 AAU/gds, 4 AAU/gds, 6 AAU/gds, 8 AAU/gds, and 10 AAU/gds was incubated with 30% ds corn endosperm at pH 5.5 and at a temperature of 60° C. During the incubation the slurry was gently stirred with an overhead mixer. After time internals of 2, 4, 6, 12 and 24 hours, the percent solubilized starch and sugar compositions (% W/W) were determined. Percent solubility, DP of sugars produced, and percentage fermentable sugars obtained were measured and calculated as described above.

### Results

TABLE 4
Solubility, DP, and percentage fermentable sugars obtained from
corn endosperm.

| AAU/gds | 2 | 4 | 6 | 8 | 10 |
|---|---|---|---|---|---|
| % Solubility | 60.3 | 68.0 | 70.8 | 73.5 | 75.1 |
| % DP1 | 58.06 | 50.12 | 48.77 | 44.38 | 44.78 |
| % DP2 | 24.89 | 26.84 | 27.29 | 28.31 | 28.10 |
| % DP3 | 9.81 | 12.93 | 13.61 | 15.42 | 15.28 |
| % DP4+ | 7.24 | 10.11 | 10.33 | 11.89 | 11.84 |
| % DP1 + DP2 | 82.93 | 76.96 | 76.06 | 72.69 | 72.88 |
| (% Fermentable | | | | | |
| sugars) | | | | | |

The results are shown in Table 4. Alpha-amylase with a concentration of 2 AAU/gds gave a higher percentage DP1 and DP2 and a lower concentration of DP3 or DP4+ than either the 8 AAU/gds or 10 AAU/gds conditions. Additionally, Alpha-amylase with a concentration of 2 AAU/gds resulted in superior overall percentage of fermentable sugars.

### Example 5

### The Effect of Humicola Glucoamylase Concentration Used in Conjunction with Alpha-Amylase on the Solubilization of Corn Endosperm Granular Starch in Corn Endosperm

This study looked at the effect of varied Humicola-glucoamylase (GA) concentrations on the solubilization and hydrolysis of corn endosperm granular starch by alpha-amylase during incubation at pH 5.5 and at a temperature below the gelatinization temperature of the starch in the corn endosperm.

### Materials and Methods

Endosperm (Valero Renewable Jefferson Plant, N5355 Junction Rd. Jefferson, and WI. 53549) was ground in a mill and then passed through sieve having a 590 micron opening. The moisture content was 14.8%. An aqueous slurry containing 30% DS was made and the pH was adjusted to 5.4. After pre-warming incubation jars for ˜30 min in the 60° C. shaking incubator, SPEZYME® RSL was dosed to all of jars at a rate of 8 AAU/g of ds endosperm and increasing dose concentrations of Humicola glucoamylase (0.025, 0.05, 0.075, 0.1 and 0.2 GAU/gds) were added the series of jars. Then, the slurry was gently shaken at 160 rpm. After 48 hours the solubility and sugar composition of the soluble fraction were determined as described in Example 1.

### Results

TABLE 5
Solubility, DP, and percentage fermentable sugars obtained from corn endosperm.

| | | 8 AAU | 8 AAU | 8 AAU | 8 AAU | 8 AAU |
|---|---|---|---|---|---|---|
| | 8 AAU | +0.025 GAU | +0.05 GAU | +0.075 GAU | +0.1 GAU | +0.2 GAU |
| % Solubility | 80.6 | 89.9 | 90.6 | 92 | 92.7 | 94.9 |
| % DP1 | 61.02 | 80.19 | 86.05 | 88.46 | 89.81 | 91.29 |
| % DP2 | 22.89 | 14.63 | 10.57 | 8.85 | 7.89 | 6.81 |
| % DP3 | 9.23 | 3.11 | 1.8 | 1.26 | 0.98 | 0.71 |
| % DP 4+ | 6.86 | 2.07 | 1.59 | 1.42 | 1.32 | 1.18 |
| % DP1 + DP2 | 83.91 | 94.82 | 96.62 | 97.31 | 97.8 | 98.1 |

The results are shown in Table 5. Both starch solubilization and production of fermentable sugar content were increased with increasing concentration of glucoamylase during incubation of endosperm with 8 AAU/gds of SPEZYME® RSL.

### Example 6

### Corn Endosperm Solubilization by Varied Glucoamylases

This study examined the effect of different glucoamylases on the solubilization and hydrolysis of corn endosperm granular starch by alpha-amylase during incubation at pH 5.5 and at a temperature below the gelatinization temperature of the starch in the corn endosperm.

### Materials and Methods

Milled Corn endosperm with a moisture content of 14.4% and screened through U.S. Standard #40 screen was weighed into different screw-capped glass jars so as to contain 50 g of slurry at 30% ds. The pH was then adjusted to 5.4-5.5.

After pre-warming incubation jars for ˜30 min at 60° C. in a shaking incubator, 8 AAU/gds of SPEZYME® RSL was added along with different glucoamylases (A. niger-GA (OPTIDEX® L-400 from Genencor Danisco), Tr-GA (GC 147 from Genencor-Danisco) and Humicola-GA (H-GA; Genencor-Danisco)) at 0.1 GAU/gds to successive jars. Then, the slurry was gently shaken at 160 rpm. After 48 hours, the percent solubilization and the sugar composition in the soluble fraction were determined as detailed in Example 1.

### Results

TABLE 6
Solubility, DP, and percentage fermentable
sugars obtained from corn endosperm.

| Gluco- | % | % | % | % | % | % Fermentables |
|---|---|---|---|---|---|---|
| amylase | Solubility | DP1 | DP2 | DP3 | DP4+ | DP1 + DP2 |
| H-GA | 92.7 | 89.81 | 7.89 | 0.98 | 1.32 | 97.70 |
| Tr-GA | 92.8 | 83.41 | 12.76 | 2.14 | 1.68 | 96.17 |
| An-GA | 92.5 | 80.09 | 14.58 | 3.19 | 2.14 | 94.67 |

The results are shown in Table 6. Humicola-GA produced 97.7% fermentable sugar compared to lower percentages of fermentable sugars obtained using other GAs.

### Example 7

### The Effect of Variable Dry Solid Concentrations on Solubility and Percent Fermentable Sugar

This experiment was conducted to compare the effect of different concentrations of dry solids (ds) in corn endosperm slurry on enzymatic breakdown of starch by alpha-amylase and GA at a temperature below the gelatinization temperature of the starch in the corn endosperm.

### Materials and Methods

The experiment was run on four different concentrations of dry solids: 30%, 32%, 34% and 36%. Respective slurries having different dry solids level were weighed and transferred to screw-capped glass jars to make a final 50 g slurry. The pH of the slurry was adjusted to pH 5.4 using 6N hydrochloric acid. SPEZYME® Xtra (12.0 AAU/gds) and H-GA (0.05 and 0.1 HAU/gds) were then added. The temperature was maintained at 60° C. During the incubation, the slurry was gently stirred in a shaking incubator. After 47 hours, the Refractive Index method was used to determine the percent of the starch solubilized and sugar compositions of the solubilized starch fraction were determined by HPLC as described in Example 1.

### Results

TABLE 7
Solubility, DP, and percentage fermentable sugars obtained from
corn endosperm having variable concentrations of dry solids.

| DS | | % | % | % | % | % | % Fermentables |
|---|---|---|---|---|---|---|---|
| (%) | Treatments (/gds) | Solubility | DP1 | DP2 | DP3 | DP4+ | (DP1 + DP2) |
| 30 | 12 AAU + | 94.3 | 85.53 | 10.89 | 1.94 | 1.65 | 96.4 |
| | 0.05 GAU | | | | | | |
| 30 | 12 AAU + | 95.8 | 89.55 | 8.1 | 1.03 | 1.31 | 97.7 |
| | 0.1 GAU | | | | | | |
| 32 | 12 AAU + | 84.1 | 86.26 | 10.35 | 1.91 | 1.48 | 96.6 |
| | 0.05 GAU | | | | | | |
| 32 | 12 AAU + | 90.2 | 89.29 | 8.24 | 1.04 | 1.43 | 97.5 |
| | 0.1 GAU | | | | | | |
| 34 | 12 AAU + | 79.6 | 86.1 | 10.5 | 1.91 | 1.49 | 96.6 |
| | 0.05 GAU | | | | | | |
| 34 | 12 AAU + | 86.2 | 89.05 | 8.28 | 1.13 | 1.54 | 97.3 |
| | 0.1 GAU | | | | | | |
| 36 | 12 AAU + | 85.3 | 84.54 | 11.59 | 2.11 | 1.76 | 96.1 |
| | 0.05 GAU | | | | | | |
| 36 | 12 AAU + | 85.3 | 88.89 | 8.49 | 1.15 | 1.47 | 97.4 |
| | 0.1 GAU | | | | | | |

The results are shown in Table 7 which indicates that solubilization of the granular starch decreased with increasing ds. However, the addition of more glucoamylase resulted in an increase in the solubilization of up to 34% ds. The highest percentage of fermentable sugars was obtained with using 30% ds and 0.1 GAU Humicola glucoamylase.

### Example 8

### Simultaneous Production of a Fermentation Feedstock and a Co-Product of Corn Gluten Fiber

This study examined the simultaneous production of fermentation feedstock and a co-product of corn gluten fiber fraction.

### Materials and Methods

Corn endosperm (Valero Renewable Jefferson Plant, N5355 Junction Rd. Jefferson Wis. 53549) was ground in an MPX mill at minimum clearance to have a particle size distribution of 1.6% on a 590 micron opening sieve, 10% on a 420 micron opening sieve and 88.4% through the 420 micron opening sieve. The moisture content was 14.2%. A twelve kg aqueous slurry containing 31.2% DS was made and the pH was adjusted to 5.4 with 6 N HCl. The slurry was then transferred to a 14 liter reactor fitted with a turbine bladed agitator driven from the bottom. The reactor was temperature controlled via heat exchange coils and an external heated circulating water bath.

The slurry was dosed with 8 AAU of SPEZYME® RSL and 0.05 GAU of HGA per gram of dry substance. The reaction was sampled for percent solubilization and saccharide composition at various times before terminating at 48 hours of reaction.

The resulting co-product insoluble fraction was separated by vacuum filtration across 24 cm #4 Whatman paper into a 4 liter side arm vacuum flask at 26-28 inches of vacuum. The resulting cake was washed with a volume of water that was approximately half the original volume of slurry and then dried in a forced draft oven at 35° C. for 40 hours. This material was ground through a falling number mill at setting #2.

Protein determination: Protein concentrations in the insoluble corn protein fiber fraction (CPFF) were determined using the Bradford QuickStart™ Dye Reagent (Bio-Rad, California, USA). For example, a 10 μL sample of the enzyme was combined with 200 10 μL Bradford QuickStart™ Dye Reagent. After thorough mixing. It was then incubated for at least 10 minutes at room temperature. Air bubbles were removed and the optical density was measured at 595 nm. The protein concentration was then calculated using a standard graph with bovine serum albumin.

### Results

TABLE 8
Solubility, dissolved solids in sugar syrup, DP, and
percentage fermentable sugars obtained from corn
endosperm sampled at various reaction times.

| | | DS | | | | | | |
|---|---|---|---|---|---|---|---|---|
| Reaction | % | of | | | | | | DP1 + |
| time | Soluble | Syrup | DP1 | DP2 | DP3 | DP4 | DP5+ | DP2 |
| 4 | 58.9 | 19.6 | 52.6 | 27.9 | 9.1 | 3.0 | 7.4 | 80.5 |
| 8 | 70.2 | 23.1 | 63.0 | 24.9 | 6.2 | 2.0 | 3.9 | 87.9 |
| 12 | 77.1 | 24.8 | 68.8 | 21.6 | 5.2 | 1.5 | 3.0 | 90.4 |
| 24 | 87.8 | 27.5 | 78.9 | 15.4 | 3.4 | 0.7 | 1.7 | 94.3 |
| 35 | 91.2 | 28.6 | 83.1 | 12.7 | 2.4 | 0.4 | 1.3 | 95.8 |
| 48 | 94.1 | 29.3 | 85.5 | 11.0 | 1.9 | 0.3 | 1.2 | 96.6 |

Table 8 shows that greater than 94% of the starch is solubilized in 48 hours and that greater than 96% fermentable are produced with the composition of 29.3% DP1+85.5% DP2. The high level of percent fermentable sugars (DP1+DP2) demonstrates the value of the endogenous hydrolyzing enzymes remaining in the corn endosperm.

TABLE 9
Composition co-products from insoluble
corn protein fiber fraction (CPFF)

| Parameter | CPFF | Endosperm | CGM | Corn | DDGS | CGF |
|---|---|---|---|---|---|---|
| % Dry Substance | 93.65 | 86.2 | 90 | 84 | 89 | 88.5 |
| % Starch (dsb) | 32.2 | 83.3 | 19 | 71.7 | | |
| % Crude Protein | 29.5 | 7.4 | 66.7 | 9.5 | 30.8 | 22.6 |
| (dsb) | | | | | | |
| % Fat (dsb) | 5.94 | 1.5 | 2.8 | 4.3 | 11.2 | 4.0 |
| % Acid detergent | 7.16 | 1.2 | 5.5 | 3.3 | 13.7 | 14.7 |
| fiber (dsb) | | | | | | |
| % Ash (dsb) | 0.43 | 0.36 | 2 | 1.4 | 5.7 | 8 |
| % Total digestible | 88.8 | 89.7 | 83.3 | | 86.4 | 90 |
| nutrients (dsb) | | | | | | |

The results of the analysis of compositions present in the insoluble corn protein fiber fraction (CPFF) are shown in Table 9. Averages values for proximate analysis of corn and corn gluten meal (CGM), corn gluten feed (CGF) are included along with the analysis of starting endosperm. Data for CGM, CGF and corn are published values by the Corn Refiners Association. Data for DDGS are defined by the American Feed Control Officials Inc., Official Publication 2007, as product obtained after the removal of ethyl alcohol by distillation from yeast fermentation of a grain or a grain mixture by condensing and drying at ¾ of solids of the resultant whole stillage using methods employed in the grain distilling industry. University of Minnesota, Department of Animal Sciences, Comparison Tables for Proximate Analysis of DDGS (March 2009).

Co-product from this process provides a gluten product containing fiber that has not been subjected to high temperatures during liquefaction as for DDGS or long periods of exposure to sulfur dioxide (SO₂) as have the feed products from corn wet milling which is associated with feed off odors. Due to the fact that most of the germ is removed during the de-germination process, the resulting percent fat level is also lower than average DDGS.

### Example 9

### Use of Different Alpha-Amylases to Degrade Starch in Corn Endosperm

This study provides a comparison of different commercially available thermostable liquefying alpha amylases on the solubilization and production of fermentable sugar during incubation of corn endosperm granular starch at a temperature below the gelatinization temperature of the starch in the corn endosperm.

### Materials and Methods

Corn endosperm (Valero Renewable Jefferson Plant, N5355 Junction Rd. Jefferson Wis. 53549) was ground in an MPX mill at minimum clearance to have a particle size distribution of 1.6% on a 590 micron opening sieve, 10% on a 420 micron opening sieve and 88.4% through the 420 micron opening sieve. The moisture content was 14.2%. Aqueous slurry containing 30% ds was made and the pH was adjusted to 5.4 with 6 N HCl. The slurry was dosed with different commercially available thermostable liquefying alpha amylases (shown in Table 9) as per manufacturer's recommended dose. The flasks were then placed in an incubated shaker maintained at 60° C. Samples were withdrawn at different intervals of time during incubation for determination of starch solubilization and HPLC composition for fermentable sugar composition as described in Example 1.

### Results

TABLE 10
Solubility, DP, and percentage fermentable sugars obtained from corn endosperm.

| | | Recommended | | | | | | | | |
|---|---|---|---|---|---|---|---|---|---|---|
| | | Dosage | Dosage | Incubation | | % | % | % | % | |
| Commercial | | for | used in the | Time | % | DP | DP | DP | DP | Fermentables |
| product | Supplier | liquefaction | study | (Hour) | Solubility | 1 | 2 | 3 | 4+ | (DP1 + DP2) |
| SPEZYME ® | Genencor | 0.2-0.5 | 0.26 | 48 | 73.6 | 59.31 | 23.61 | 9.53 | 7.55 | 82.9 |
| Xtra | | KG/MTds | KG/MTds | | | | | | | |
| | | corn | corn | | | | | | | |
| SPEZYME ® | Genencor | 0.2-0.5 | 0.26 | 48 | 64 | 69.07 | 19.83 | 6.14 | 4.97 | 88.9 |
| Alpha | | KG/MTds | KG/MTds | | | | | | | |
| | | corn | corn | | | | | | | |
| SPEZYME ® | Genencor | 0.2-0.5 | 0.26 | 48 | 65.8 | 66.78 | 21.26 | 6.78 | 5.17 | 88.0 |
| RSL | | KG/MTds | KG/MTds | | | | | | | |
| | | corn | corn | | | | | | | |
| Liquozyme ® | Novozymes | 0.2-0.5 | 0.26 | 48 | 43.5 | 76.79 | 15.13 | 3.69 | 4.39 | 91.9 |
| SC DS | | KG/MTds | KG/MTds | | | | | | | |
| | | corn | corn | | | | | | | |
| Fuelzyme ® | Verenium | 0.2-0.5 | 0.30 | | | | | | | |
| LF | | KG/MTds | KG/MTds | 48 | 24.8 | 71.72 | 11.75 | 3.38 | 13.15 | 83.5 |
| | | corn | corn | | | | | | | |

As seen from the results in Table 10, addition of different commercial liquefying alpha amylases resulted in various level of granular starch solubilization. However, all alpha amylases tested produced high levels of fermentable sugar. Without being limited by theory, it is hypothesized that the difference in the fermentable sugar content could be due to the differences in the availability of soluble substrate to endogenous starch hydrolyzing enzymes. For example, lower solubilization generally results in higher percentage of DP1 and DP2.

### Example 10

### Use of AMY E in the Solubilization of Corn Endosperm

This example examines the effect of AMY E, an endo-acting liquefying and saccharifying alpha amylase, incubated with SPEZYME® RSL on starch solubilization and production of fermentable sugar at a temperature below the gelatinization temperature of the starch in the corn endosperm.

### Materials and Methods

Corn endosperm (Valero Renewable Jefferson Plant, N5355 Junction Rd. Jefferson Wis. 53549) was ground in an MPX mill at minimum clearance to have a particle size distribution of 1.6% on a 590 micron opening sieve, 10% on a 420 micron opening sieve, and 88.4% through the 420 micron opening sieve. The moisture content was 14.2%. Aqueous slurry containing 30% ds was made and the pH was adjusted to 5.4 with 6 N HCl. SPEZYME® RSL was added at 6.0 AAU/gds. The slurry was dosed with different amounts of AMY E (01, 0.5 and 1.0 mg of AMY E per gds) The flasks were placed in an incubated shaker maintained at 60° C. Samples were withdrawn at different intervals of time during incubation for determining the starch solubilization and HPLC composition for fermentable sugar composition as described in Example 1.

### Results

TABLE 11
Solubility, DP, and percentage fermentable
sugars obtained from corn endosperm.

| | | | | | | % |
|---|---|---|---|---|---|---|
| | % | % | % | % | % | Fermentables |
| Enzymes | Solubility | DP1 | DP2 | DP3 | DP4+ | (DP1 + DP2) |
| No AmyE | 74.7 | 57.41 | 24.90 | 10.43 | 7.26 | 82.3 |
| added | | | | | | |
| 0.1 mg | 85.0 | 49.64 | 27.58 | 13.21 | 9.57 | 77.2 |
| AME | | | | | | |
| Protein/gds | | | | | | |
| 0.5 mg | 88.0 | 60.16 | 23.97 | 9.31 | 6.56 | 84.1 |
| AME | | | | | | |
| Protein/gds | | | | | | |
| 1.0 mg | 90.5 | 63.99 | 22.53 | 7.88 | 5.6 | 86.5 |
| AMY E | | | | | | |
| Protein/gds | | | | | | |

The data in Table 11 shows that the endo-acting liquefying and saccharifying alpha amylase AMY E in combination with SPEZYME® Xtra during the incubation of corn endosperm resulted in high solubilization of the granular corn starch and production of fermentable sugars at levels greater than 85%.

### Example 11

### Comparison of Commercially Available Corn Endosperms for the Production of Fermentable Sugars

This study examined commercially available corn endosperms for their potential to produce fermentable sugars at temperatures below the gelatinization temperature of the starch in the grain.

### Materials and Methods

Corn endosperms are currently produced on a commercial scale either for food applications or fuel alcohol production using dry fractionation processes (Alexander, 1987, “Corn Dry Milling: Process, Products, and Applications,” in Corn Chemistry and Technology, (Watson & Ramstead, eds.; American Association of Cereal Chemists, Inc.), pgs. 351-376), or wet fractionation (U.S. Pat. No. 6,566,125). Both processes produce endosperm containing greater than 80% starch on a dry weight basis. Comparative studies were conducted by making an aqueous slurry containing 30% ds using endosperm (ground and passed through 40-mesh sieve) from two different manufactures: 1) Valero Renewable Jefferson Plant, Jefferson, Wis.; and 2) ICM, Colwich, Kans.

After the pH of the slurry was adjusted to 5.4, SPEZYME® XTRA was added at 8 AAU/gds and then incubated at 60° C. for 48 hours. Samples were taken at different intervals time to determine the percent solubilization of the corn granular starch and sugar composition of fermentable sugars as described in Example 1.

### Results

TABLE 12
Solubility, DP, and percentage fermentable
sugars obtained from different sources of corn endosperm.

| Manufacturer | H-GA | Hour | % Solubility | % DP1 | % DP2 | % DP3 | % DP4+ | % fermentable sugars |
|---|---|---|---|---|---|---|---|---|
| Valero | 0 | 24 | 70.1 | 52.6 | 26.39 | 11.98 | 9.03 | 79.0 |
| | +H-GA | | 79.6 | 80.66 | 14.08 | 2.89 | 2.38 | 94.7 |
| | 0 | 48 | 75.0 | 65.25 | 22.36 | 7.64 | 4.76 | 87.6 |
| | +H-GA | | 84.9 | 87.35 | 9.61 | 1.60 | 1.45 | 87.0 |
| ICM | 0 | 24 | 64.0 | 65.77 | 21.05 | 7.37 | 5.82 | 86.8 |
| | +H-GA | | 74.8 | 86.08 | 10.29 | 1.59 | 2.04 | 96.4 |
| | 0 | 48 | 70.4 | 74.9 | 16.82 | 4.72 | 3.57 | 91.7 |
| | +H-GA | | 81.1 | 90.06 | 7.48 | 0.86 | 1.6 | 97.5 |

As shown in Table 12, both commercially available samples of corn endosperm produced high levels of fermentable sugars during incubation with SPEZYME® XTRA and Humicola-GA.

### Example 12

### Direct Starch to Fermentable Sugar Using Rice Flour

This example looked at production of DSTFS syrup using an alternative source of starch for starch degradation at temperatures below that of the gelatinization temperature of starch in the grain.

### Materials and Methods

An aqueous slurry (30% ds) of rice flour was prepared and the pH was adjusted to pH 5.5. SPEZYME® XTRA and two different glucoamylase preparations were added at different doses and incubated at 60° C. with constant mixing. Samples were taken at different intervals of time during incubation and percent rice starch solubilized and sugar composition of the soluble fractions were determined as described in Example 1.

TABLE 13
DP and percentage of starch solubilized
using rice flour as a starch source.

| | | | | | | % |
|---|---|---|---|---|---|---|
| | | | | | | starch |
| | | % | % | % | % | solubi- |
| Treatment | Hrs | DP1 | DP2 | DP3 | DP4+ | lized |
| Control | 19 | 41.125 | 32.552 | 18.121 | 8.202 | 80.01 |
| (SPEZYME ® | 26 | 42.758 | 32.019 | 17.475 | 7.749 | 82.73 |
| XTRA 10 | 43 | 48.000 | 29.648 | 15.510 | 6.842 | 89.67 |
| AAU/gds) | 48 | 49.463 | 28.984 | 14.941 | 6.611 | 89.96 |
| SPEZYME ® | 19 | 69.008 | 22.500 | 6.992 | 1.500 | 83.28 |
| XTRA 10 U/gds + | 26 | 72.679 | 20.431 | 6.889 | 0.000 | 84.37 |
| DISTILLASE | 43 | 78.058 | 17.017 | 4.925 | 0.000 | 90.52 |
| ASP, 0.1 GAU/gds | 48 | 79.345 | 16.128 | 4.527 | 0.000 | 91.94 |
| SPEZYME ® | 19 | 82.423 | 14.346 | 3.231 | 0.000 | 87.71 |
| 10 AAU/gds + | 26 | 84.641 | 12.747 | 2.612 | 0.000 | 92.51 |
| DISTILLASE ® ASP, | 43 | 87.276 | 10.684 | 2.039 | 0.000 | 93.66 |
| 0.3 GAU/gds | 48 | 87.630 | 10.213 | 2.157 | 0.000 | 94.81 |
| SPEZYME ® | 19 | 86.166 | 11.601 | 2.233 | 0.000 | 90.52 |
| XTRA 10 AAU/gds + | 26 | 87.475 | 10.623 | 1.902 | 0.000 | 97.13 |
| DISTILLASE ® ASP, | 43 | 88.813 | 9.541 | 1.646 | 0.000 | 99.77 |
| 0.5 GAU/gds | 48 | 88.976 | 9.399 | 1.626 | 0.000 | 98.59 |
| SPEZYME ® | 19 | 75.259 | 18.316 | 6.425 | 0.000 | 87.43 |
| XTRA | 26 | 78.749 | 16.188 | 5.063 | 0.000 | 90.24 |
| 10 AAU/gds + | 43 | 82.668 | 13.568 | 3.764 | 0.000 | 93.08 |
| H-GA −0.1 GAU/gds | 48 | 83.430 | 13.012 | 3.558 | 0.000 | 94.81 |
| SPEZYME ® | 19 | 87.523 | 10.326 | 2.151 | 0.000 | 88.27 |
| XTRA | 26 | 89.018 | 9.308 | 1.674 | 0.000 | 91.94 |
| 10 AAU/gds + H-GA, | 43 | 90.239 | 8.345 | 1.416 | 0.000 | 94.23 |
| 0.3 GAU/gds | 48 | 90.471 | 8.181 | 1.349 | 0.000 | 96.26 |
| SPEZYME ® | 19 | 90.793 | 7.927 | 1.280 | 0.000 | 93.08 |
| XTRA, | 26 | 91.297 | 7.621 | 1.081 | 0.000 | 95.97 |
| 10 AAU/gds + H-GA, | 43 | 91.425 | 7.514 | 1.061 | 0.000 | 98.89 |
| 0.5 GAU/gds | 48 | 91.379 | 7.570 | 1.052 | 0.000 | 99.47 |

The results in Table 13 show the complete solubilization of the rice starch at high concentrations of glucoamylase with 10 AAU of SPEZYME® XTRA and the solubilized starch contained greater than 98% DP1 and DP2 sugars.

### Example 13

### Direct Starch to Fermentable Sugar Using Whole Ground Wheat

This example describes the direct conversion of wheat granular starch to fermentable sugars production using whole ground wheat as a substrate.

### Materials and Methods

Whole ground wheat (32% ds) was hydrolyzed using only SPEZYME™ XTRA (B. stearothermophilus alpha-amylase (AA)) in one experiment and 2 combinations of SPEZYME™ XTRA and Humicola-glucoamylase (GA) in another. Sugar profiles were obtained from the hydrolysis of whole ground wheat by endogenous wheat enzymes, alpha-amylase, and/or combinations of AA/GA. The Alpha-Amylase was dosed at 9 AAU/gr ds and the Humicola GA either at 0.05 GAU/gr ds or 0.1 GAU/gr ds. A negative control was also measured to monitor the endogenous enzyme activity of wheat. Conditions were 32% DS, 60° C., pH 5.2, 9 AAU/g ds SPEZYME™ XTRA; 9 AAU/g ds SPEZYME™ XTRA and 0.05 GAU/gr ds Humicola GA; 9 AAU/g ds SPEZYME™ XTRA and 0.1 GAU/gr ds Humicola GA; or endogenous wheat enzymes (negative control), respectively, for each experimental trial run. Samples were withdrawn at different time intervals during the hydrolysis for HPLC analysis and the measurement of starch solubilization as described in Example 1.

### Results

TABLE 14
Solubility, DP, and percentage fermentable sugars obtained.

| Pretreatment | Hour | % Starch Solubility | % DP1 | % DP2 | % DP3 | % DP4+ | % fermentable sugars |
|---|---|---|---|---|---|---|---|
| 9 AAU/g | 0 | 16.7 | 23.8 | 17.6 | 0.9 | 57.7 | 41.4 |
| SPEZYME ™ | 5 | 89.3 | 3.8 | 58.7 | 10.7 | 26.8 | 62.5 |
| XTRA | 21.5 | 96.3 | 5.0 | 61.6 | 15.7 | 17.7 | 66.6 |
| | 27 | 96.1 | 5.3 | 62.0 | 16.6 | 16.2 | 67.3 |
| | 44.5 | 99.8 | 5.6 | 62.7 | 18.4 | 13.3 | 68.3 |
| | 48 | 99.8 | 5.5 | 62.9 | 18.7 | 12.9 | 68.4 |
| 9 AAU/g | 0 | 14.7 | 25.0 | 14.6 | 1.4 | 59.0 | 39.6 |
| SPEZYME ™ | 5 | 90.1 | 23.2 | 50.6 | 3.1 | 23.1 | 73.8 |
| XTRA + | 21.5 | 94.5 | 57.3 | 27.6 | 3.6 | 11.6 | 84.9 |
| 0.05 GAU/g | 27 | 97.0 | 63.5 | 22.9 | 3.7 | 9.9 | 86.4 |
| Humicola GA | 44.5 | 94.8 | 77.4 | 11.9 | 3.8 | 6.9 | 89.3 |
| | 48 | 98.6 | 78.9 | 10.6 | 3.8 | 6.7 | 89.5 |
| 9 AAU/g | 0 | 15.6 | 23.5 | 17.1 | 1.8 | 57.5 | 40.6 |
| SPEZYME ™ | 5 | 90.7 | 19.7 | 53.1 | 3.6 | 23.6 | 72.8 |
| XTRA + | 21.5 | 96.9 | 49.3 | 34.2 | 3.6 | 12.9 | 83.5 |
| 0.1 GAU/g | 27 | 97.6 | 55.1 | 29.9 | 3.8 | 11.2 | 85 |
| Humicola GA | 44.5 | 95.0 | 68.8 | 18.9 | 4.1 | 8.2 | 87.7 |
| | 48 | 100.0 | 71.0 | 17.2 | 4.1 | 7.8 | 88.2 |
| Endogenous | 0 | 16.6 | 22.9 | 21.0 | 1.5 | 54.6 | 43.9 |
| wheat | 5 | 74.8 | 3.6 | 50.7 | 3.9 | 41.8 | 54.3 |
| enzymes | 21.5 | 79.8 | 4.5 | 50.9 | 7.5 | 37.1 | 55.4 |
| (negative | 27 | 79.6 | 4.8 | 51.2 | 8.2 | 35.8 | 56 |
| control) | 44.5 | 82.9 | 4.5 | 50.9 | 9.8 | 34.9 | 55.4 |
| | 48 | 80.3 | 4.5 | 50.9 | 9.9 | 34.7 | 55.4 |

The data in Table 14 demonstrates that, with the single addition of a high dosage of alpha-amylase, more than 65% of fermentable sugars (DP1+DP2) are produced after 21.5 hrs. Starch solubility is greater than 95% after 21.5 hrs of hydrolysis.

The addition of 0.05 GAU/g ds Gluco-Amylase in combination with the AA results in greater than 80% of fermentable sugars after 21.5 hrs and greater than 89% fermentable sugars after 44.5 hrs. Starch hydrolysis is greater than 95% after 27 hrs of hydrolysis.

Where Gluco-Amylase levels were increased to 0.1 GAU/g ds, greater than 80% fermentable sugars were released after 21.5 hrs and greater than 88% after 48 hrs. Starch hydrolysis is greater than 95% after 21.5 hrs of hydrolysis. The negative control experiment shows that endogenous wheat enzymes are capable of releasing 55% of the fermentable sugars after 21.5 hrs and obtaining a starch hydrolysis of greater than 80% after 44.5 hrs.

### Example 14

### Direct conversion of barely granular starch to fermentable sugars

This Example describes the direct conversion of barley granular starch to fermentable sugars production using whole barley as a substrate at a temperature below that of the gelatinization temperature of the starch in the barley.

Whole barley is steeped and then milled and an aqueous slurry containing 32% ds is prepared. The pH of the slurry is adjusted to pH 5.25 and incubated with only SPEZYME™ XTRA (B. stearothermophilus AA) in one experiment and a combination of SPEZYME™ XTRA and Humicola-GA in another. The Alpha-Amylase is dosed at 8 AAU/gr ds and the Humicola-GA at 0.05 GAU/gr ds. The hydrolysis is performed at 60° C. and pH 5.25. Samples are withdrawn at different time intervals during the hydrolysis for HPLC analysis and the measurement of starch solubilization as described in Example 1.

### Example 15

### Fermentation of Feedstock Produced by DSTFS

This example examined the fermentation of sugar syrups produced using the method described in Example 7 to produce ethanol as a fermentation product.

### Materials and Methods

Sugar syrup produced according to the process described in Example 7 was thawed in a 75° C. water bath and allowed to cool to room temperature. The syrup was then adjusted to a final dry solids content of 22% but had corn steep liquor added at 0.1% solids. Urea (1,200 ppm) and corn steep liquor (0.1% ds) were added as nutrients for the yeast used for the fermentation. Another flask was set up without added corn steep liquor as control. The pH of the medium was adjusted to pH 5.0 and inoculated with active yeast (Ethanol Red, Red Star Yeast). Additional glucoamylase was also added at 0.1 GAU/gds (DISTILLASE® SSF from Genencor, Danisco). Yeast fermentation was carried out by incubating at 32° C., and shaken at 150 rpm. Samples were taken during different intervals of time and analyzed by HPLC.

TABLE 15
Yeast fermentation of feedstock produced by DSTFS into ethanol.

| | | | % | % | % | % | % | % | % | % |
|---|---|---|---|---|---|---|---|---|---|---|
| | | | W/V | W/V | W/V | W/V | W/V | W/V | W/V | V/V |
| Flask | Description | hrs | DP > 3 | DP-3 | DP-2 | DP-1 | Lactic | Glycerol | Acetic | Ethanol |
| 1, 2 | Fermentable | 0 | 0.254 | 0.158 | 0.565 | 22.057 | 0.000 | 0.000 | 0.000 | 0.000 |
| 1, 2 | syrup | 16 | 0.116 | 0.180 | 0.919 | 21.209 | 0.000 | 0.199 | 0.141 | 0.740 |
| 1, 2 | Urea | 24 | 0.018 | 0.051 | 0.580 | 19.656 | 0.089 | 0.354 | 0.143 | 1.149 |
| 1, 2 | | 40 | 0.019 | 0.054 | 0.582 | 18.611 | 0.000 | 0.421 | 0.157 | 1.592 |
| 1, 2 | | 48 | 0.012 | 0.049 | 0.547 | 17.096 | 0.000 | 0.419 | 0.141 | 1.582 |
| 3, 4 | Fermentable | 0 | 0.254 | 0.158 | 0.565 | 22.057 | 0.000 | 0.000 | 0.000 | 0.000 |
| 3, 4 | syrup | 16 | 0.006 | 0.052 | 0.562 | 20.355 | 0.075 | 0.250 | 0.165 | 0.747 |
| 3, 4 | GA | 24 | 0.000 | 0.051 | 0.561 | 19.541 | 0.000 | 0.324 | 0.162 | 1.160 |
| 3, 4 | Urea | 40 | 0.007 | 0.055 | 0.550 | 18.518 | 0.067 | 0.483 | 0.158 | 1.586 |
| 3, 4 | | 48 | 0.011 | 0.053 | 0.541 | 17.900 | 0.000 | 0.460 | 0.159 | 1.684 |
| 5, 6 | Syrup + GA + liquor | 0 | 0.355 | 0.169 | 0.579 | 22.636 | 0.326 | 0.000 | 0.000 | 0.000 |
| 5, 6 | Syrup + GA + liquor | 16 | 0.119 | 0.092 | 0.725 | 8.730 | 0.396 | 0.759 | 0.000 | 7.488 |
| 5, 6 | Syrup + GA + liquor | 24 | 0.060 | 0.057 | 0.598 | 0.095 | 0.310 | 1.027 | 0.000 | 12.585 |
| 5, 6 | Syrup + GA + liquor | 40 | 0.044 | 0.038 | 0.468 | 0.011 | 0.067 | 1.026 | 0.079 | 12.767 |
| 5, 6 | Syrup + GA + liquor | 48 | 0.019 | 0.033 | 0.541 | 0.011 | 0.040 | 1.027 | 0.105 | 12.773 |

The results are shown in Table 15. High fermentable sugar syrup was unable to support the growth of yeast during fermentation with only added urea as nutrient. However, the addition of corn steep liquor resulted in complete conversion of fermentable sugars into ethanol in 24 hours of the fermentation. Addition of glucoamylase did not show any additional benefits in terms of ethanol yield.

### Example 16

### Conversion of DSTFS Feed Stock in Lactic Acid Fermentation

This example used sugar syrup from whole ground corn produced using the direct starch to fermentable sugars (DSTFS) process as a feedstock for fermentation of the syrup into lactic acid.

### Materials and Methods

The DSTFS feed stock was prepared by incubating an aqueous slurry of whole ground corn (18% ds) at pH 5.0 with SPEZYME® ALPHA (8.0 AAU/gds) and Humicola-GA (0.1 GAU/gds) at 60° C. for 45 hours. The slurry was then centrifuged to separate the insoluble solids. The soluble solids fraction contained 96.5% fermentable sugars and was used in lactic acid fermentation. The lactic acid fermentation was carried out using Lactobacillus rhamnosus strain obtained from China General Microbiological Culture Collection Center.

Seed Medium:

Casein 10.0 g, Beef extract 10.0 g, Yeast extract 5.0 g, Glucose 5.0 g, Sodium acetate 5.0 g, Diammonium citrate 2.0 g, Tween 80 1.0 g, K₂HPO₄ 2.0 g, MgSO₄ 7 H2O, 0.2 g, MnSO₄*H₂O 0.05 g, Agar 15.0 g, Distilled water 1.0 L, pH 6.8. To one thousand grams of fermentation feed stock in a two liter fermenter, the following nutrients were added: 40 g corn steep liquor, 10 g casein, 10 g yeast extract, 10 g beef extract, 1.5 g Tween 80, 0.3 g MgSO₄*7H2O, 20 g CaCO₃.

The inoculums of Lactobacillus rhamnosus were transferred to 100 mL seed culture and cultivated at 37° C. at 200 rpm. The cultivation was controlled based on OD600 (absorbance) value increasing up 0.5.

50 mL of seed culture was added to a 2 L fermenter. The fermentation was carried out at a constant pH 6.5 using 20% NH₄OH and the temperature was set at 45° C., and the agitation was 200 rpm. Samples were taken for HPLC analysis at 4 hr, 21 hr, 27 hr, and 44 hr. The fermentation was also carried out by adding additional glucoamylase (0.1 GAU/gds). Lactic acid content was calculated according to lactic acid standard concentration as shown in Table 17.

### Results

TABLE 16
Production of lactic acid using a DSTFS feedstock. Concentrations are in g/L.

| Sample | | Ferm. Time | DP3+ | DP3 | DP2 | Glucose | Lactic Acid |
|---|---|---|---|---|---|---|---|
| # | Fermentations | Hour | grams/Liter | grams/Liter | grams/Liter | grams/Liter | grams/Liter |
| | DSTFS | 0 | 3.4 | 1.8 | 11.5 | 158.5 | 0 |
| 1 | DSTFS | 4 | 3.8 | 1.9 | 9.1 | 141.1 | 10.07 |
| | DSTFS + GA | | 3.1 | 1.8 | 5.6 | 137.1 | 10.07 |
| 2 | DSTFS | 21 | 4.5 | 3.3 | 8.6 | 40.1 | 94.1 |
| | DSTFS + GA | | 3.6 | 1.7 | 7.3 | 88.5 | 53.81 |
| 3 | DSTFS | 27 | 5.7 | 3.6 | 8.3 | 20.6 | 111.13 |
| | DSTFS + GA | | 5.2 | 1.7 | 9 | 50.2 | 88.47 |
| 4 | DSTFS | 44 | 5.7 | 3.6 | 7.2 | 0 | 123.24 |
| | DSTFS + GA | | 5.5 | 1.8 | 8.5 | 0.9 | 128.57 |

Results are shown in Table 16. The fermentable sugar syrup produced by the DSTFS process yielded greater than 123 grams per liter lactic acid. Addition of glucoamylase during fermentation resulted in higher level of lactic acid compared to control without added glucoamylase.

### Example 17

### Conversion of DSTFS Feedstock into Succinic Acid

This example used sugar syrup from whole ground corn produced using the direct starch to fermentable sugars (DSTFS) process as a feedstock for fermentation of the syrup into succinic acid.

The fermentable sugars feed stock used in this example is prepared as explained in Example 13 by incubating an aqueous slurry of whole ground corn (18% ds) at pH 5.0 with SPEZYME ALPHA 9.8 AAU/gds and Humicola-GA (0.1 GAU/gds) at 60° C. for 45 hours. The slurry is then centrifuged to separate the insoluble solids. The soluble fraction contained 96.5% fermentable sugars and is then used in Succinic acid fermentation.

Succinic acid fermentation utilized Actinobacillus succinogenes 130Z (ATCC 55618) from China General Microbiological Culture Collection Center. The TSB medium is inoculated with the bacterial strain in cooked meat medium. The culture incubated at 37° C. for 8 hr. Then, the activated strain in TSB medium is inoculated in seed medium, which is incubated at 37° C. for 16 hr under anaerobic conditions. Finally, after adding MgCO₃ powder the seeds strain is inoculated in fermentation medium. The fermentation is conducted under anaerobic conditions with CO₂ atmosphere for 48 hr. Fermentation broth is analyzed by HPLC, illustrating the successful generation of succinic acid.

### Example 18

### Effect of Alpha-Amylase Concentration on the Production of Kojibiose and Nigerose

The experiments in this example were conducted to demonstrate production of kojibiose and nigerose by exogenous alpha-amylase with whole ground corn.

### Materials and Methods

Aqueous whole ground corn was mixed with DI water in order to produce a 32% dry solids slurry and adjusted to pH 5.6 with HCl. The slurry (150 g) was then placed in 4 different 250 ml plastic bottles and incubated in a shaker (210 rpm) at 60° C. with 4, 8, 12 or 16 AAU/gds of SPEZYME® XTRA.

Solubilization/Saccharification was carried out up to 48 hours and samples from each jar were periodically drawn. Samples were then centrifuged to obtain supernatant for RI (Refractive Index) for calculating percent solubility and diluted by taking 0.5 ml and combining it with 4.5 ml of RO water. This was then filtered through 0.45 μm Whatman filters and put into vials for HPLC analysis. The HPLC analysis was conducted using a Rezex RCM-Monosaccharide column with a guard column for sugar composition. Further analysis for DP2 composition was carried out by capillary electrophoresis, which was confirmed by NMR (not shown).

### Results

TABLE 17
Concentration of DP2 saccharides following solubilization with alpha-amylase at
temperatures below the initial gelatinization temperature of the grain.

| SPEZYME ® | | % | | | | | | | | |
|---|---|---|---|---|---|---|---|---|---|---|
| XTRA | % starch | dry | % sugar composition | | | | DP2 grams/100 gram dry solids | | | |
| (AAU/gds) | solubilized | solids | DP1 | DP2 | DP3 | DP4+ | Maltose | Isomaltose | Kojibiose | Nigerose |
| 4 | 72.9 | 24.9 | 67.86 | 20.48 | 6.75 | 4.91 | 10.74 | 3.35 | 4.14 | 2.25 |
| 8 | 80.3 | 26.8 | 65.17 | 21.70 | 7.72 | 5.42 | 9.64 | 4.37 | 5 | 2.69 |
| 12 | 84 | 27.8 | 63.83 | 22.37 | 8.55 | 5.58 | 9.97 | 4.2 | 5.36 | 2.84 |
| 16 | 86.4 | 28.3 | 62.59 | 22.95 | 8.70 | 5.76 | 11.36 | 3.71 | 5.05 | 2.83 |

This experiment shows that higher concentrations of alpha-amylase solubilized more starch in corn, generating high levels of DP1 and DP2. In all cases, significant amount of Kojibiose and Nigerose were successfully produced. The results suggest that endogenous starch hydrolyzing enzyme in corn can produce glucose as well as the disaccharides isomaltose, kojibiose and nigerose from solubilized starch.

### Example 19

### Effect of Temperature on the Production of Kojibiose and Nigerose

This example shows the temperature effect on the hypothesized endogenous plant starch hydrolyzing enzymes in whole ground corn.

### Materials and Methods

Aqueous whole ground corn slurry was prepared by mixing with DI water in order to contain 32% dry solids and adjusted to pH 5.6 with HCl. Each 150 g of the slurry was then placed in 4 different 250 ml plastic bottles and placed either in a top-stirring waterbath at 70° C. or 83° C. or a shaker at 50° C. or 60° C. with 10 AAU/gds of SPEZYME® XTRA. After a 2 hour incubation, the slurries were put into a 60° C. shaking incubator to continue the reaction, while the slurries at 50° C. and 60° C. were maintained without temperature shift.

Solubilization/Saccharification was carried out up to 48 hours and samples from each jar were periodically drawn. Samples were then centrifuged to obtain supernatant for RI (Refractive Index) for calculating percent solubility and diluted by taking 0.5 ml and combining it with 4.5 ml of RO water. This was then filtered through 0.45 μm Whatman filters and put into vials for HPLC analysis. The HPLC analysis was conducted using a Rezex RCM-Monosaccharide column with a guard column for sugar composition. Further analysis for DP2 composition was carried out by capillary electrophoresis, which was confirmed by NMR (not shown).

### Results

TABLE 18
Concentration of DP2 saccharides following solubilization with
alpha-amylase at varied temperatures.

| | | | % | | | | | | | | |
|---|---|---|---|---|---|---|---|---|---|---|---|
| Temp | SPEZYME ® | % starch | dry | % sugar composition | | | | DP2 grams/100 gram dry solids | | | |
| ° C. | XTRA | solubilized | solids | DP1 | DP2 | DP3 | DP4+ | Maltose | Isomaltose | Kojibiose | Nigerose |
| 50 | 10 AAU/gds | 44.6 | 16.7 | 84.38 | 7.27 | 1.54 | 6.82 | 3.2 | 2.75 | 0.9 | 0.42 |
| 60 | 10 AAU/gds | 81 | 27 | 65.17 | 21.85 | 7.80 | 5.19 | 12.78 | 3.19 | 3.81 | 2.07 |
| 70 | 10 AAU/gds | 68.9 | 23.8 | 19.32 | 30.65 | 23.63 | 26.40 | 28.21 | 0.34 | 1.22 | 0.88 |
| 83 | 10 AAU/gds | 100 | 31.57 | 2.43 | 5.55 | 6.82 | 85.19 | 5.31 | 0.06 | 0.09 | 0.09 |

These data show that significant amounts of kojibiose/nigerose was successfully produced by solubilizing granular starch of corn with SPEZYME® XTRA. These data also show that the hypothesized endogenous enzymes are most active at 60° C. Temperatures at 70° C. and higher appear to inactivate the enzymes, leaving significantly lower level of kojibiose and nigerose. The productivity of these DP2 appeared to be not dependent on the concentration of AAU.

### Example 20

### DP2 Compositions from Different Grains

This example examined the ability of different small grains to produce Kojibiose/Nigerose.

### Materials and Methods

Aqueous slurries of 5 different ground grains: (A) whole ground corn, (B) dehulled ground milo, (C) ground wheat, (D) ground barley and (E) rye flour, were prepared by mixing with DI water in order to produce a 32% dry solids slurry and adjusted to pH 5.6 for milo and pH 5.9 for wheat, barley and rye with HCl, respectively. Then, each slurry (150 g) from the respective grain was placed in 8 different 250 ml plastic bottles and incubated in the shaker at 60 C with 10 AAU/gds of SPEZYME® XTRA dose. In the cases of wheat, barley and rye, 0.05 GAU/gds of H-GA was dosed in addition because a high level of maltose was expected to be produced.

Solubilization/Saccharification was carried out up to 48 hours and samples from each jar were periodically drawn. Samples were then centrifuged to obtain supernatant for RI (Refractive Index) for calculating percent solubility and diluted by taking 0.5 ml and combining it with 4.5 ml of RO water. This was then filtered through 0.45 μm Whatman filters and put into vials for HPLC analysis. The HPLC analysis was conducted using a Rezex RCM-Monosaccharide column with a guard column for sugar composition. Further analysis for DP2 composition was carried out by capillary electrophoresis, which was confirmed by NMR (not shown).

### Results

TABLE 19
Concentration of DP2 saccharides following solubilization
of different grains with alpha-amylase.

| SPEZYME ® | | | | | | | | | | | |
|---|---|---|---|---|---|---|---|---|---|---|---|
| XTRA | | % starch | % dry | % sugar composition | | | | DP2 grams/100 gram dry solids | | | |
| AAU/gds | Substrates | solubilized | solids | DP1 | DP2 | DP3 | DP4+ | Maltose | Isomaltose | Kojibiose | Nigerose |
| 10 | Corn | 81 | 27 | 65.17 | 21.85 | 7.80 | 5.19 | 12.78 | 3.19 | 3.81 | 2.07 |
| 10 | Milo | 83 | 28.1 | 70.24 | 18.42 | 6.39 | 4.94 | 1.55 | 8.08 | 1.99 | 1.02 |
| 10 | Wheat | 88.7 | 27.3 | 6.09 | 70.96 | 12.63 | 10.13 | 70.96 | ND | ND | ND |
| Add H-GA | Wheat | 94.8 | 28.7 | 78.21 | 11.42 | 3.77 | 6.60 | 11.42 | ND | ND | ND |
| (0.05 GAU/gds) | | | | | | | | | | | |
| 10 | Barley | 86.1 | 26.6 | 9.18 | 51.00 | 12.42 | 27.41 | 51 | ND | ND | ND |
| Add H-GA | Barley | 95.5 | 28.7 | 73.26 | 9.99 | 4.29 | 12.46 | 9.99 | ND | ND | ND |
| (0.05 GAU/gds) | | | | | | | | | | | |
| 10 | Rye | 93.2 | 30.67 | 10.40 | 45.68 | 17.28 | 26.64 | 45.68 | ND | ND | ND |
| Add H-GA | Rye | 95.8 | 31.29 | 75.50 | 7.93 | 4.96 | 11.60 | 7.93 | ND | ND | ND |
| (0.05 GAU/gds) | | | | | | | | | | | |

ND = Not detectable

These data indicate that only corn and milo contain the hypothesized endogenous plant starch hydrolyzing enzymes to produce unique DP2s such as Isomaltose, Kojibiose and Nigerose, whereas other grains (wheat, barley and rye) only produced maltose, presumably driven by endogenous beta-amylase.

### Example 21

### Production of Kojibiose/Nigerose from Granular Starch Using Extracts from Whole Ground Corn Slurry

This example was conducted to demonstrate extractability of endogenous enzymes from whole ground corn and their capability to produce Kojibiose and Nigerose from refined corn starch.

### Materials and Methods

Aqueous whole ground corn slurry was prepared by mixing with DI water in order to form a slurry with 30% dry solids and adjusted to pH 5.6 with HCl. Then each 150 g of the slurry was placed in a plastic bottle and let them undergo at 37° C. in the shaker. After a 3 hour incubation, the slurries were centrifuged to remove heavy solids and the resulting supernatant (extract) was recovered. 10% dry solids refined corn starch slurry was then prepared using the supernatant to incubate at 55° C. in a shaking incubator with and without 10 AAU/gds of SPEZYME® XTRA. The pH of the slurry was adjusted to 5.6 before dosing enzyme. Incubation was carried out up to 47 hours and samples were taken at 24 and 47 hours.

Samples were then centrifuged to obtain supernatant for RI (Refractive Index) for calculating percent solubility and diluted by taking 0.5 ml and combining it with 4.5 ml of RO water. This was then filtered through 0.45 μm Whatman filters and put into vials for HPLC analysis. The HPLC analysis was conducted using a Rezex RCM-Monosaccharide column with a guard column for sugar composition. Further analysis for DP2 composition was carried out by capillary electrophoresis.

### Results

TABLE 20
Concentration of DP2 saccharides following solubilization
of refined corn starchs lurry.

| | | % | | | | | | | | | | |
|---|---|---|---|---|---|---|---|---|---|---|---|---|
| | SPEZYME ® | dry | % sugar composition | | | | | | DP2 grams/100 gram dry solids | | | |
| Hour | XTRA | solids | Fructose | Unknown | DP1 | DP2 | DP3 | DP4+ | Maltose | Isomaltose | Kojibiose | Nigerose |
| 24 | No enzyme | 4.3 | 26.68 | 2.14 | 54.07 | 3.24 | 7.16 | 5.43 | Not measurable | | | |
| 47 | No enzyme | 3.7 | 24.34 | 2.68 | 51.71 | 3.97 | 9.60 | 7.71 | | | | |
| 24 | 10 AAU/gds | 13.0 | 3.67 | 0.39 | 49.08 | 23.72 | 9.77 | 13.38 | 71 | 15 | 7 | 8 |
| 47 | 10 AAU/gds | 13.1 | 3.45 | 0.69 | 64.62 | 18.63 | 6.64 | 5.85 | 51 | 24 | 11 | 14 |

As observed in Table 20, a significant amount of kojibiose/nigerose was successfully produced by solubilizing granular starch of corn with whole ground corn slurry extract and SPEZYME® XTRA, while there was negligible amount of kojibiose/nigerose when SPEZYME XTRA® added. The results showed that endogenous enzyme is soluble can be extracted with water. Additionally, the results indicate that water-extractable endogenous enzyme is capable of producing kojibiose and nigerose.

### Example 22

### Effect of pH Treatment of Whole Ground Corn Slurry on Reduction of Kojibiose/Nigerose

This example was conducted to demonstrate the effect of pH on endogenous plant starch hydrolyzing enzymes in whole ground corn and to propose a method to inactivate endogenous enzymes.

### Materials and Methods

Aqueous whole ground corn slurry was prepared by mixing with DI water in order to contain 32% dry solids. The slurry was then split into 2 groups: one is maintained at pH 5.5 and the other adjusted to pH 3.0 with HCl, followed by incubation at 60° C. for 1 hour in the shaking incubator. The slurry without pH adjustment is incubated at 60° C. in a shaking incubator, while the slurry at pH 3.0 was pH-adjusted back to pH 5.6 to continue the incubation.

The reaction was carried out up to 22 hours and samples were then centrifuged to obtain supernatant for RI (Refractive Index) for calculating percent solubility and diluted by taking 0.5 ml and combining it with 4.5 ml of RO water. This was then filtered through 0.45 μm Whatman filters and put into vials for HPLC analysis. The HPLC analysis was conducted using a Rezex RCM-Monosaccharide column with a guard column for sugar composition. Further analysis for DP2 composition was carried out by capillary electrophoresis.

### Results

TABLE 21
Concentration of DP2 saccharides following solubilization
with and without pre-pH treatment.

| | | | | % | | | | | | | | |
|---|---|---|---|---|---|---|---|---|---|---|---|---|
| pH pre- | SPEZYME ® | | % starch | dry | % sugar composition | | | | DP2 grams/100 gram dry solids | | | |
| treatment | XTRA | H-GA | solubilized | solids | DP1 | DP2 | DP3 | DP4+ | Maltose | Isomaltose | Kojibiose | Nigerose |
| No | 10 AAU/gds | 0.1 GAU/gds | 79.6 | 26.1 | 87.34 | 9.32 | 1.36 | 1.97 | 27.4 | 26.3 | 31.5 | 14.8 |
| 3.0 | 10 AAU/gds | 0.1 GAU/gds | 79.3 | 25.1 | 90.55 | 2.94 | 1.55 | 4.96 | 89.0 | 11.0 | 0 | 0 |

The data demonstrate that endogenous enzymes were inactivated during pre-treatment at pH 3.0 in terms of formation of kojibiose and nigerose. Additionally, the results show that the resulting syrup contains higher DP1 and lower DP2, providing more fermentability.

### Example 23

### Use of DSTFS Feedstock for Industrial Enzyme Production

This example used sugar syrup from whole ground corn produced using the direct starch to fermentable sugars (DSTFS) process as a feedstock for the industrial production of protease and phytases enzymes.

### Materials and Methods

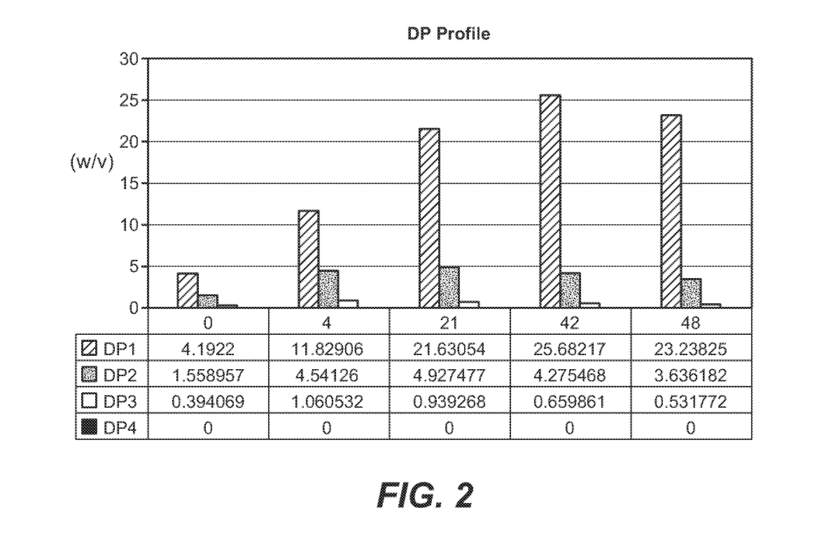

35% of corn slurry was prepared by mixing the ground corn and process water and incubated for 1 hour at room temperature. pH was adjusted the corn slurry to pH 5.3 to 5.5 using sulfuric acid. 0.37 g/kg of SPEZYME RSL and 0.09 g/kg of GC321 was added to the in corn slurry, and then the temperature was increased to 60C for 48 hour incubation. pH was maintained between 5.3 to 5.5. The BP sugar profile of the sugar syrup was determined using the methods described above and is illustrated in FIG. 2.

Heat evaporation was used for syrup concentration, and the concentrated syrup was harvested and cooled for enzyme fermentation.

### Results

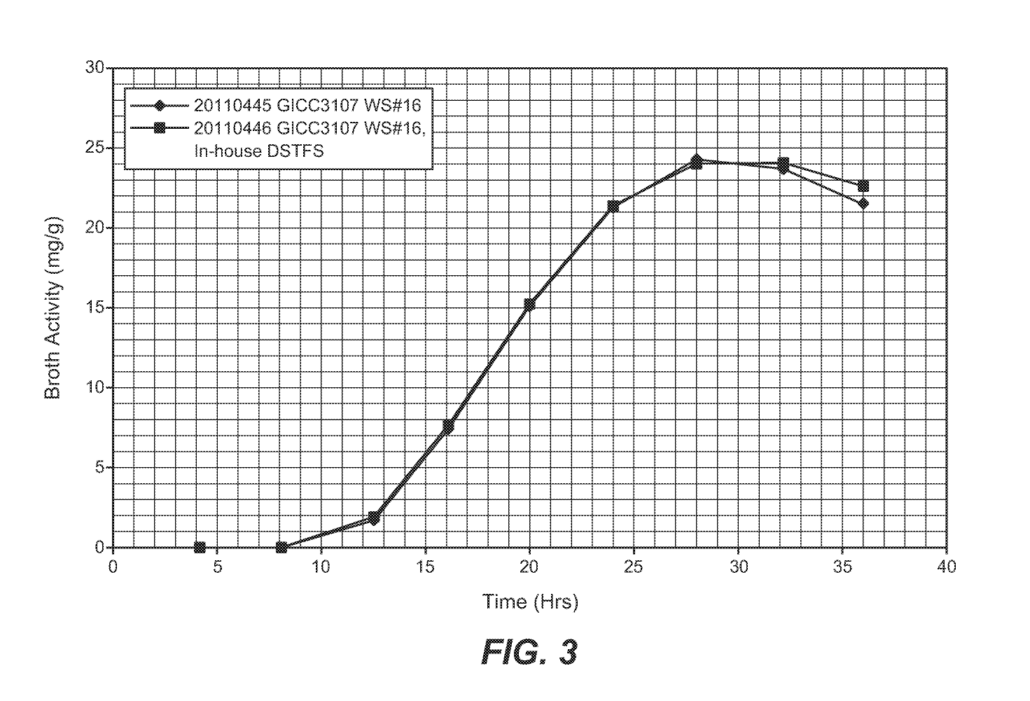

Several types of fermentations were run and validated using self-made corn syrup (DSTFS) and compared to conventional industrial sugars. FIG. 3 shows the enzyme activity vs. fermentation time for a representative experiment illustrating generation of protease activity from Bacillus amyloliquefaciens expressed in Bacillus subtilis (FNA).

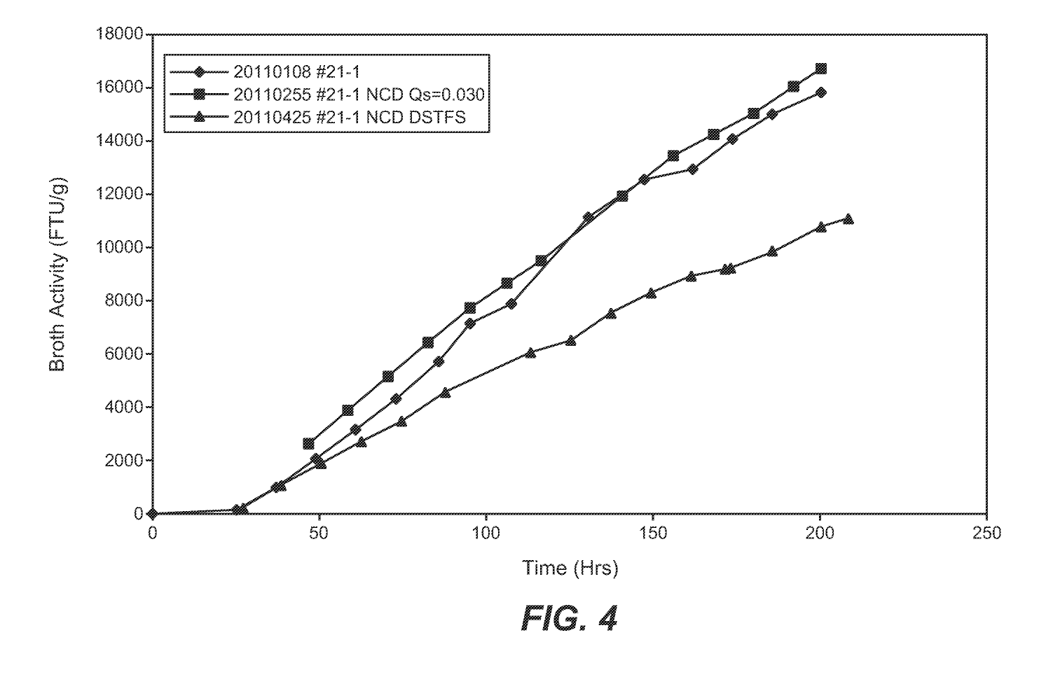

Another representative experiment is shown in FIG. 4 illustrated the generation of BP111 phytase activity (derived from Buttiauxella) expressed in Trichoderma reseei.

This example shows that sugars produced by the DSTFS process can successfully be used for the industrial production of enzymes.

### Example 24

### Effects of Phytase Addition to Corn in a Granular Starch Hydrolysis (GSH) Process

This example was conducted to determine if phytase addition to corn in a granular starch hydrolysis (GSH) process with alpha amylase and gluco-amylase will increase starch solubilization.

In a GSH process, the goals are to have ideally 100% solubilization of starch and produce >95% glucose. Generally, phytase may be added to the liquefaction process to hydrolyze phytic acid. When phytic acid is hydrolyzed, alpha amylase may work better and/or there may be more starch that becomes available for enzymes. In addition, phytic acid decreases alpha amylase activity at high temperature by chelating calcium, sodium, and other ions. With the natural amount of phytic acid present in corn, it is thought that by hydrolyzing phytic acid with the addition of phytase in a GSH process, the solubilization may increase.

### Materials and Methods

The materials for this example were: (1) 32% ds ground corn; (2) 32% ds corn flour slurry; (3) SPEZYME Xtra enzyme (Sticker #2010-0556 and activity 13,249 AAUs/g); (4) BP111 enzyme (Sticker #2009-0005 and activity 63,480 FTUs/g); (5) HGA enzyme (Sticker #2009-1615 and activity 440 GAUs/g); and (6) 25% v/v NH₄OH, 6N HCl.

Whole ground corn of 500 g at 32% ds was made with DI water. The slurry was mixed and the pH was adjusted to 5.4 using 25% v/v ammonium hydroxide. Then, 200 grams of the slurry were poured into 2 separate 250 mL flasks. The two runs were dosed with 1) 10 AAUs Xtra and 0.2 GAUs HGA, and 2) 10 AAUs Xtra+10 FTUs BP111+0.2 GAUs HGA. The samples were placed in shakers at 60° C. and 200 rpm for 48 hours. All samples were run in duplicate, and an additional run was completed with 17 AAUs Xtra+0.2 GAUs HGA to determine if a high amount of alpha amylase would achieve the same solubilization (but at faster rate) as a run with a lower amount of alpha amylase.

Samples were taken at 3, 20, 26, and 48 hours for saccharide distribution. A sample was measured into a 2.0 mL centrifuge tube and centrifuged at 13.2 k rpm for approximately 4 minutes. After centrifugation, the refractive index of the supernatant was determined at 30° C. The remaining supernatant was filtered into a separate centrifuge tube through a 3 mL syringe with a 0.45 μm GHP membrane and boiled for 10 minutes to terminate the alpha amylase activity. The boiled sample was prepared for HPLC analysis, where 0.5 mL of filtrate was mixed with 4.5 mL of RO water and placed into HPLC vials.

Samples were taken for complete solubility testing any time after 24 hours from the start of the experiment. This was done by adding 1 drop of SPEZYME FRED into a 2.0 mL centrifuge tube and filling the rest of the tube with sample. The sample was then boiled for 10 minutes and the refractive index was determined at 30° C. The total dry solids are determined by this refractive index. Solubility was determined by taking the supernatant % ds divided by the total % ds and multiplied by 100.

### Results and Discussion

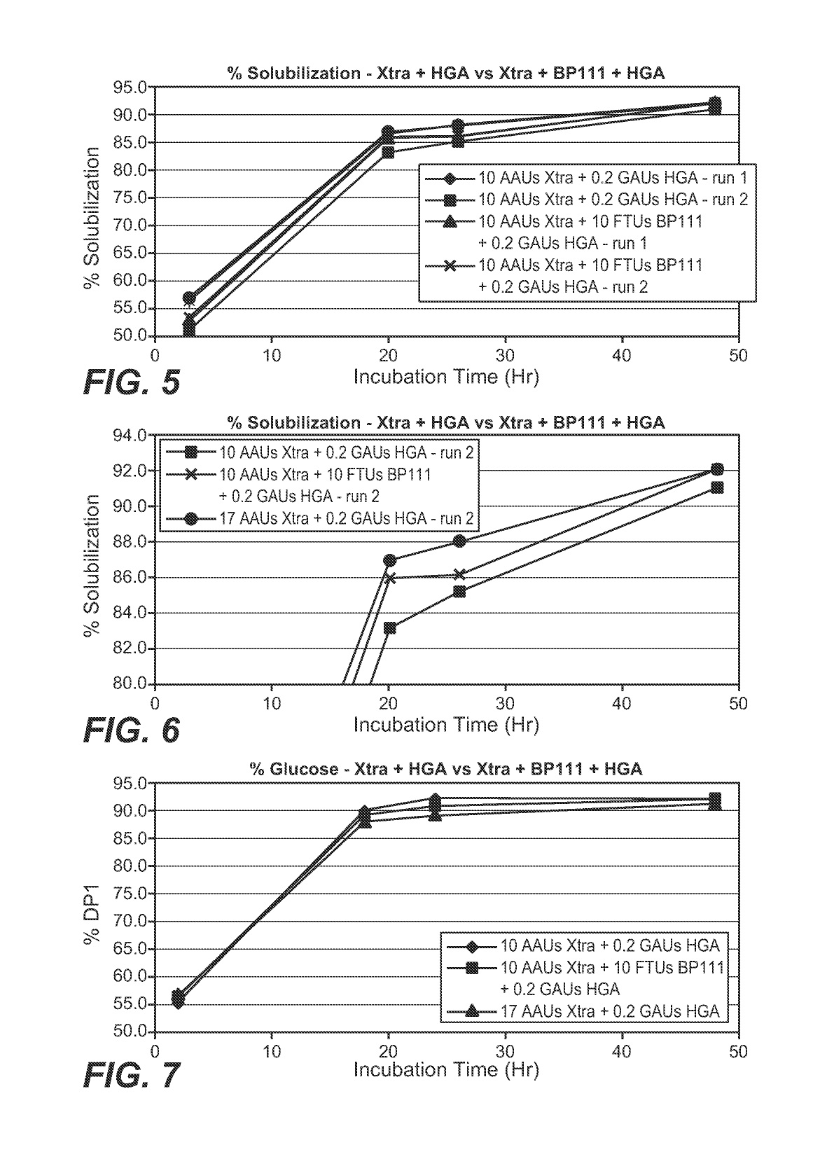

FIGS. 5 and 6 show the % solubilization of the three different runs at 60° C. FIG. 5 shows the % solubilization over 48 hours for the GSH process. Table 22 summarizes the % DP1 and solubilization results for the GSH process.

As can be seen from FIG. 6, the run with 17 AAUs Xtra+0.2 GAUs HGA and the run with 10 AAUs Xtra+10 FTUs BP111+0.2 GAUs HGA reached the same percent solubilization of 92.1 at 48 hours. This was higher than 10 AAUs Xtra+0.2 GAUs HGA run by 1%.

Results on the increased solubilization with phytase are not conclusive since there could be two reasons for the higher solubility: 1) alpha amylase works better when phytic acid is hydrolyzed, or 2) more starch becomes available for enzymes to work on from breaking the phytic acid and starch complex. This experiment should be repeated with a GA only GSH process to remove alpha-amylase as a dependent variable.

TABLE 22
The % DP1 and solubilization results for the GSH process

| | | % | % |
|---|---|---|---|
| Name | hr | DP1 | Solubilization |
| 10 AAUs Xtra + 0.2 GAUs HGA - run 2 | 3 | 63.387 | 51.2 |
| | 20 | 83.664 | 83.2 |
| | 26 | 85.324 | 85.2 |
| | 48 | 86.214 | 91.1 |
| 10 AAUs Xtra + 10 FTUs BP111 + 0.2 | 3 | 61.458 | 53.3 |
| GAUs HGA - run 2 | 20 | 83.412 | 85.9 |
| | 26 | 84.56 | 86.2 |
| | 48 | 85.85 | 92.1 |
| 17 AAUs Xtra + 0.2 GAUs HGA - run 2 | 3 | 60.478 | 57.0 |
| | 20 | 82.387 | 87.0 |
| | 26 | 83.064 | 88.0 |
| | 48 | 85.82 | 92.1 |

### Example 25

### Effects of Phytase Addition in a Granular Starch Hydrolysis (GSH) Process

This example was conducted in order to determine if phytase addition in a granular starch hydrolysis process will increase the solubilization of starch.

In a GSH process, the goals are to have as close to 100% solubilization of starch and produce >95% glucose. Generally, phytase may be added to the liquefaction process to hydrolyze phytic acid. When phytic acid is hydrolyzed, the alpha amylase enzyme may work better and/or there may be more starch that becomes available for enzymes. In addition, phytic acid decreases alpha amylase activity at high temperature by chelating calcium, sodium, and other ions.

A model system was prepared with starch and added phytic acid as substrate for GSH. With an addition of phytic acid to starch in this example, it was hypothesized that hydrolyzing phytic acid with addition of phytase in a GSH process may increase the solubilization. One experiment was performed with alpha amylase and gluco-amylase while a second experiment was performed with only gluco-amylase to remove alpha-amylase as the dependent factor.

### Materials and Methods

The materials for this example were: (1) Gel starch (89.15% ds); (2) Phytic acid sodium salt hydrate; (3) SPEZYME Xtra enzyme sticker (Sticker #2010-0556 and activity 13,249 AAUs/g); (4) BP111 enzyme (Sticker #2009-0005 and activity 63,480 FTUs/g); (5) HGA enzyme (Sticker #2009-1615 and activity 440 GAUs/g); and (6) 25% v/v NH₄OH, 6N HCl.

### GSH Process Using Alpha Amylase and Gluco-Amylase

Corn has around 1% phytic acid on a dry basis. A model system with starch +1% phytic acid was tested for effect on solubility. The main objective was to determine if increased solubility comes from enhanced alpha amylase activity or from breaking the phytic acid and starch complex. To test this, a GSH process with only gluco-amylase was performed where alpha amylase was not added to the starch slurry.

Raw starch was made up to 400 grams with DI water at 32% ds. Then, 1% phytic acid salt on a dry basis was added to starch and incubated overnight on a stir plate at room temperature to allow phytic acid to bind to starch. After 24 hours of mixing, the starch sample was adjusted to pH 5.4 with 25% v/v ammonium hydroxide. Then, 100 grams of the sample were poured into 3 separate 100 mL bottles, with extra starch left over. Each 100 gram sample was dosed separately with: 1) 10 AAUs SPEZYME Xtra+0.2 GAUs HGA; 2) 10 AAUs Xtra+10 FTUs BP111+0.2 GAUs HGA; and 3) 17 AAUs Xtra+0.2 GAUs HGA. The samples were placed in 60° C. water bath and stirred for 48 hours.

Samples were taken at 2, 18, 24, and 48 hours for saccharide distribution. A sample was measured into a 2.0 mL centrifuge tube and centrifuged at 13.2 k rpm for approximately 4 minutes. After centrifugation, the refractive index of the supernatant was determined at 30° C. The remaining supernatant was filtered into a separate centrifuge tube through a 3 mL syringe with a 0.45 μm GHP membrane and then boiled for 10 minutes to terminate the amylase activity. The boiled sample was prepared for HPLC analysis, where 0.5 mL of filtrate was mixed with 4.5 mL of RO water and placed into HPLC vials.

Samples were taken for complete solubility testing any time after 24 hours from the start. This was done by adding 1 drop of SPEZYME FRED into a 2.0 mL centrifuge tube and filling the rest of the tube with sample. It was then boiled for 10 minutes and the refractive index was determined at 30° C. The total dry solids are determined by this refractive index. Solubility was determined by taking the supernatant % ds divided by the total % ds and multiplied by 100.

### GSH Process Using Only Gluco-Amylase

The objective of this example was to study the effect of additional 5% phytic acid on starch solubility compared to a GA only GSH. Then it was tested if the solubility would increase if phytase is also added with phytic acid.

Starch slurries were prepared with and without phytic acid. A 200 g, 32% ds starch slurry was made up and mixed overnight on stir plate with 5% phytic acid. Another set of starch slurries were prepared at 32% ds without phytic acid. All of the starch slurries were adjusted to ph 5.4 with 25% v/v ammonium hydroxide.

The starch slurry with 5% phytic acid was dosed with and without phytase: 1) 1 GAU HGA, and 2) 1 GAU HGA+10 FTUs BP111. The starch slurry without any phtyic acid was dosed with 1 GAU HGA only. The samples were then placed in 60° C. water bath and stirred for 48 hours.

Samples were taken at 4, 25, 30, and 48 hours for percent solubilization. A sample was measured into a 2.0 mL centrifuge tube and centrifuged at 13.2 k rpm for approximately 4 minutes. After centrifugation, the refractive index of the supernatant was determined at 30° C. Samples were also taken for complete solubility testing any time after 24 hours from the start of the experiment. This was done by adding 1 drop of SPEZYME FRED into a 2.0 mL centrifuge tube and filling the rest of the tube with sample. It was then boiled for 10 minutes and the refractive index was determined at 30° C. The total dry solids are determined by this refractive index. Solubility was determined by taking the supernatant % ds divided by the total % ds and multiplied by 100.

### Results and Discussion

GSH Process with Alpha Amylase and Gluco-Amylase

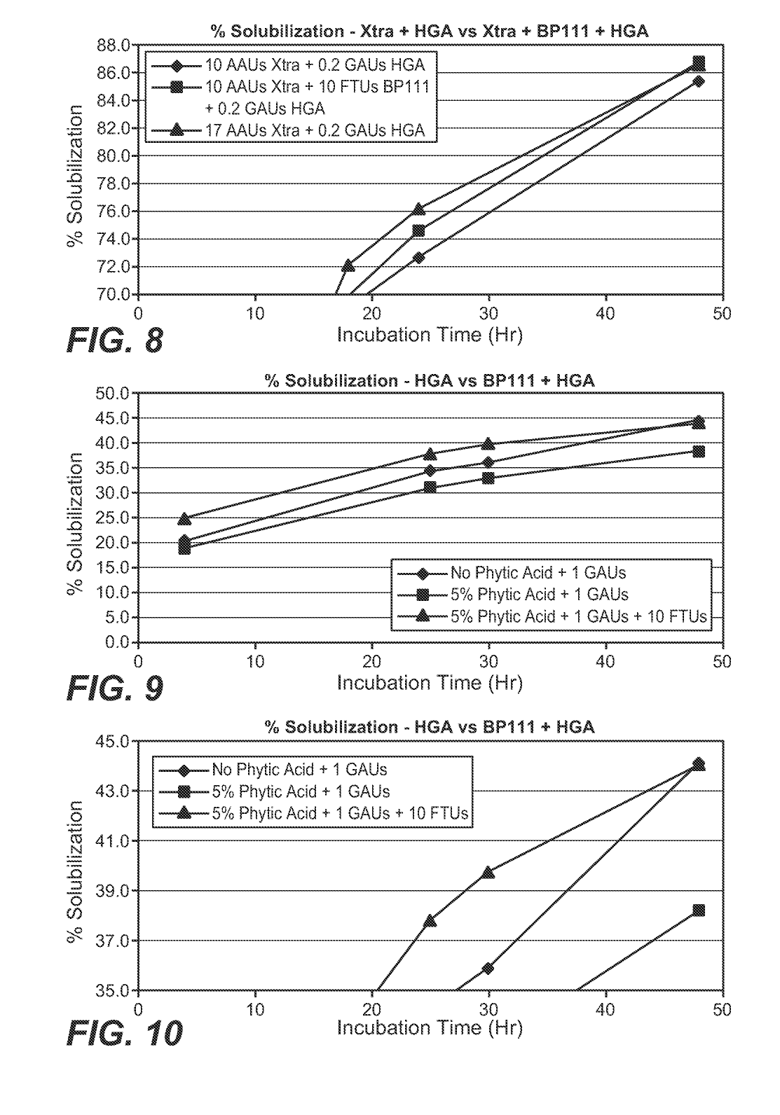

FIGS. 7 and 8 show the % solubilization of the three different runs at 60° C. As can be seen from FIG. 8, the run with 17 AAUs Xtra+0.2 GAUs HGA and the run with 10 AAUs Xtra+10 FTUs BP111+0.2 GAUs HGA reached approximately the same percent solubilization of 86.6-86.8 at 48 hours. This was higher than the 10 AAUs Xtra+0.2 GAUs HGA run by 1.2-1.4%. The glucose levels for all three runs went to 91.2-92.0%. Table 25 summarizes the % solubilization and DP1 results.

Results on the increased solubilization with phytase are not conclusive since there may be two reasons for the higher solubility: 1) alpha amylase works better when phytic acid is hydrolyzed, or 2) more starch becomes available for enzymes to work on from breaking the phytic acid and starch complex. Accordingly, the next part of the example was performed to determine the reasons for increased solubilization with phytase.

TABLE 23
The % DP1 and solubilization for GSH process with alpha amylase
and glucoamylase

| | | % | % |
|---|---|---|---|
| Name | hr | DP1 | Solubilization |
| 10 AAUs Xtra + 0.2 GAUs HGA | 2 | 55.19 | 40.68 |
| | 18 | 89.99 | 69.09 |
| | 24 | 92.01 | 72.87 |
| | 48 | 91.21 | 85.43 |
| 10 AAUs Xtra + 10 FTUs BP111 + 0.2 | 2 | 56.50 | 39.18 |
| GAUs HGA | 18 | 88.82 | 69.92 |
| | 24 | 90.68 | 74.57 |
| | 48 | 91.96 | 86.80 |
| 17 AAUs Xtra + 0.2 GAUs HGA | 2 | 57.08 | 42.20 |
| | 18 | 87.95 | 72.23 |
| | 24 | 89.00 | 76.30 |
| | 48 | 91.19 | 86.57 |

GSH Process with Gluco-Amylase

FIGS. 9 and 10 show the % solubilization of the three different runs at 60° C. As shown in the two graphs, the run with no phytic acid +1 GAUs HGA and the run with 5% phytic acid +1 GAUs HGA+10 FTUs BP111 both reached same percent solubilization at the end of 48 hours with 44.1%. This was 5.9% higher solubilization compared to the run with 5% phytic acid+1 GAUs HGA (no phytase addition). Higher solubilization achieved with phytase addition allowed for more starch to become available by hydrolyzing phytic acid that is bound to starch.

What is claimed is:

1. A method of making a fermentable sugar feedstock, the method comprising treating an aqueous slurry of ground or fractionated grain with an alpha amylase and a glucoamylase to produce the fermentable sugar feedstock;
wherein the treatment is at a temperature at or below the initial gelatinization temperature of the starch in the grain;
wherein the concentration of the alpha amylase is between about 5 to about 20 AAU/gds; and
wherein the fermentable sugar feedstock comprises a higher concentration of DP-2 saccharides in comparison to fermentable sugar feedstocks that are not made by treating an aqueous slurry of ground or fractionated grain with a starch solubilizing alpha amylase and a glucoamylase, wherein the treatment is at a temperature below the initial gelatinization temperature of the grain, and wherein the concentration of the alpha amylase is between about 5 to about 20 AAU/gds.

2. The method of claim 1, wherein the treatment is at a temperature of about 0 to about 30° C. below the initial gelatinization temperature of the starch in the grain.

3. The method of claim 1, wherein the concentration of alpha amylase is about 6 AAU/g ds to about 10 AAU/g ds.

4. The method of claim 1, wherein the ground or fractionated grain is selected from the group consisting of: corn, corn endosperm, milo, rice, and any combination thereof.

5. The method of claim 1, wherein greater than about 90% of the starch from the ground or fractionated grain is solubilized.

6. The method of claim 5, wherein the solubilized starch comprises greater than about 90% fermentable sugars.

7. The method of claim 1, wherein the alpha amylase is derived from a Bacillus spp.

8. The method of claim 1, wherein the alpha amylase is selected from the group consisting of SPEZYME® XTRA, SPEZYME® Alpha, SPEZYME® RSL, Liquozyme SC, and Fuelzyme.

9. The method of claim 1, further comprising treating the aqueous slurry with one or more enzymes selected from the group consisting of: cellulases, hemicellulases, pullulanases, pectinases, phytases, and proteases.

10. The method claim 1, further comprising treating the aqueous slurry with an acid fungal alpha amylase.

11. The method of claim 1, wherein the treatment is at a temperature of about 55 to about 65° C.

12. The method of claim 1, wherein the concentration of glucoamylase is about 0.025 GAU/g ds to about 0.075 GAU/g ds.

13. The method of claim 1, wherein the concentration of glucoamylase is about 0.075 GAU/g ds to about 0.2 GAU/g ds.

14. The method of claim 1, wherein the DP-2 saccharides comprise kojibiose and/or nigerose.

15. The method of claim 1, further comprising using the fermentable sugar feedstock as a carbon source for the industrial production of one or more products by a fermenting microorganism.

16. The method of claim 15, wherein the fermenting microorganism is a yeast or a bacteria.

17. The method of claim 15, wherein the product is an enzyme.

18. The method of claim 17, wherein the enzyme is used in the processing of grain, as an additive or in the preparation of food, as an additive or in the preparation of animal feed, as an ingredient in a detergent or cleaning agent, in the processing of textiles, or in the processing of pulp for the manufacture of paper.

19. The method of claim 15, wherein the product is a fermentation product selected from the group consisting of ethanol, lactic acid, gluconic acid, butanol, succinic acid, citric acid, monosodium glutamate, sodium gluconate, calcium gluconate, potassium gluconate, glucono delta lactone, sodium erythorbate, itaconic acid, ketones, amino acids, glutamic acid (sodium monoglutamate), penicillin, tetracyclin, enzymes, vitamins, and hormones.

20-72. (canceled)
